(19) United States
(12) Patent Application Publication
(10) Pub. No.: US 2016/0115455 A1
(43) Pub. Date: Apr. 28, 2016

(54) REPROGRAMMED CELLS AND METHODS OF PRODUCTION AND USE THEREOF

(71) Applicants: THE BROAD INSTITUTE, INC., Cambridge, MA (US); PRESIDENT AND FELLOWS OF HARVARD COLLEGE, Cambridge, MA (US)

(72) Inventors: Tarjei Mikkelsen, Cambridge, MA (US); Davide Cacchiarelli, Cambridge, MA (US)

(21) Appl. No.: 14/891,897

(22) PCT Filed: May 16, 2014
(86) PCT No.: PCT/US14/38491

§ 371 Date: Nov. 17, 2015

Related U.S. Application Data
(60) Provisional application No. 61/824,918, filed on May 17, 2013.

(51) Int. Cl. C12N 5/074; G01N 33/50

(52) U.S. Cl. CPC C12N 5/0696; G01N 33/5073; C12N 2501/602; C12N 2501/606; C12N 2501/603; C12N 2501/604

(57) ABSTRACT

The present invention relates generally to a secondary reprogramming technique and its uses. More particularly, it concerns immortalized secondary somatic cells, secondary induced pluripotent stem cells (iPSCs), secondary induced multipotent progenitor cells (iMPCs) and tertiary somatic cells derived therefrom, and methods of using these cells to assess the effects of agents on cells and for medical treatment of subjects.

### RELATED APPLICATIONS

This application claims priority from U.S. Provisional Application Ser. No. 61/824,918, filed May 17, 2013, the contents of which are incorporated herein by reference in its entirety.

### GOVERNMENT SUPPORT

This invention was made with government support under Grant Number PO1GM099117 awarded by NIH/NIGMS. The government has certain rights in the invention.

### FIELD OF THE INVENTION

The present invention relates generally to a secondary reprogramming technique and its uses. More particularly, it concerns immortalized secondary somatic cells, secondary induced pluripotent stem cells (iPSCs), secondary induced multipotent progenitor cells (iMPCs) and tertiary somatic cells derived therefrom, and methods of using these cells to assess the effects of agents and for the treatment of subjects.

### BACKGROUND

Nuclear reprogramming is an approach to generate embryonic-like stem cells from somatic cells by the ectopic expression of defined pluripotency factors (see, e.g., K. Takahashi and S. Yamanaka (2006) Cell, 126: 663-676; K. Takahashi et al. (2007) Cell, 131: 861-872; J. Yu et al. (2007) Science, 318: 1917-1920; M. Nakagawa et al. (2008) Nat. Biotechnol., 26: 101-106; International Publication WO 2007/069666; and International Publication WO2010/068955). Generation of the resulting induced pluripotent stem cells (iPSCs) is a long, highly inefficient process affected by the predisposition of the starting somatic cell to be reprogrammed and the stoichiometry of reprogramming factors. Conventional systems employing direct infection of somatic cells with viral vectors expressing the four pluripotency factors Oct4, Klf4, Myc, and Sox2 are extremely inefficient, typically yielding ˜0.2% pluripotent cells, and this efficiency is even lower in human cell cultures. The subsequent development of drug-inducible secondary systems sought to remedy these reprogramming inefficiencies (D. Hochemeyer et al. (2008) Cell Stem Cell, 3:346-353 and N. Maherali et al. (2008) Cell Stem Cell, 3: 340-345). These secondary systems consist of fibroblasts differentiated from iPSCs obtained through drug-induced expression of a construct containing the pluripotency factors. These secondary fibroblasts represent a genetically homogeneous starting material from which secondary iPSCs can be generated. However, its use only marginally improves reprogramming efficiencies to up to ˜5%. Existing secondary systems suffer from several limitations, mainly related to cellular senescence, which affects the consistency of derivation and reprogramming of secondary somatic cells.

The lack of robust methodologies to reprogram somatic cells and to generate high-quality and uniform iPSCs hinders the widespread use of iPSCs and cells derived therefrom in high-throughput screening, genome editing-based studies, and therapeutics.

### SUMMARY OF THE INVENTION

The present invention provides for a secondary reprogramming technique, as well as cells produced at various stages along the production pathway. Such cells include i) immortalized secondary somatic cells containing an inducible polycistronic exogenous nucleic acid encoding reprogramming factor(s), wherein the immortalized secondary somatic cells are derived either directly from an initial cell, or indirectly via a de-differentiated cell, such as a primary induced pluripotent stem cell (iPSC)), ii) secondary de-differentiated cells (such as secondary iPSCs), and iii) tertiary somatic cells derived from immortalized secondary somatic cells either directly, or indirectly via a secondary iPSC or other de-differentiated intermediate. Reprogramming a somatic cell includes altering its differentiation state, such as by de-differentiating the somatic cell. In certain embodiments, reprogramming comprises reprogramming to a pluripotent state (e.g., generating an iPSC). In such embodiments, the reprogramming factor(s) may also be referred to as pluripotency factor(s). In other embodiments, reprogramming comprises de-differentiating the cell to a multipotent cell that is not pluripotent (e.g., the cell is de-differentiated but does not pass through a pluripotency stage). Exemplary multipotent cells are multipotent progenitor cells and, in certain embodiments, secondary induced MPCs (iMPCs) are provided.

In certain embodiments, such cells include i) immortalized secondary somatic cells containing an inducible polycistronic exogenous nucleic acid encoding reprogramming factor(s), which immortalized secondary somatic cells were derived either directly from an initial cell that de-differentiates but does not pass through a pluripotent state (e.g., the initial cell is reprogrammed without passing through pluripotency, to a primary iMPC), or indirectly via a de-differentiated cell that passes through pluripotency (e.g., the initial cell is reprogrammed to a pluripotent state, such as a primary iPSC), ii) secondary de-differentiated cells (such as secondary iPSCs or secondary iMPCs), and iii) tertiary somatic cells derived from immortalized secondary somatic cells either directly via a de-differentiated intermediate that does not pass through pluripotency, e.g., a secondary iMPC, or indirectly via a de-differentiated intermediate that does pass through pluripotency, e.g., a secondary iPSC.

In one aspect, the invention provides for a cell that i) contains an inducible first polycistronic exogenous nucleic acid encoding reprogramming factor(s) operably linked to a first regulatory sequence, and ii) expresses a second exogenous nucleic acid encoding an immortalizing factor operably linked to a second regulatory sequence. In certain embodiments, the cell does not express the reprogramming factors encoded in the inducible first polycistronic exogenous nucleic acid (i.e., an immortalized secondary somatic cell). Such cells may be reprogrammed by inducing expression of the reprogramming factors (produced from the exogenous nucleic acid, such as a viral vector present in the cell and capable of expressing the nucleic acid). In certain embodiments, immortalized secondary somatic cells may be reprogrammed by inducing expression of the reprogramming factors encoded on the first inducible exogenous nucleic acid. In other embodiments, immortalized secondary somatic cells may be reprogrammed by inducing expression of the reprogramming factors encoded on a third exogenous nuclei acid. In yet another embodiment, immortalized secondary somatic cells may be reprogrammed by exposing the cells to an agent(s) that induces endogenous expression of a reprogramming factor(s).

The invention provides a cell produced by inducing the expression of a reprogramming factor(s) in such an immortalized secondary somatic cell, wherein the immortalized secondary somatic cell of a first cell type is directly reprogrammed into a somatic cell of a second cell type, without passing through a discrete pluripotency stage (e.g., tertiary somatic cells). Optionally, such an immortalized secondary somatic cell passes through a de-differentiated intermediate, for example, an iMPC intermediate.

The present invention also provides for secondary somatic cells that contain a first exogenous nucleic acid encoding an immortalizing factor and a second exogenous inducible nucleic acid encoding a reprogramming factor(s), ii) secondary de-differentiated cells (such as secondary iMPCs) derived thereof, and iii) tertiary somatic cells derived from such immortalized secondary somatic cells directly via a de-differentiated intermediate that does not pass through pluripotency, e.g., a secondary iMPC. The disclosure provides methods for generating these cell types and for using these cells in various methods, such as assay methods. An example of this is set forth in FIG. 1B and in Example 5.

In certain embodiments, a de-differentiated intermediate is a transient cell state, rather than a distinct, stable cell type.

In certain embodiments, the reprogramming factors are selected for their ability to induce pluripotency (i.e., pluripotency factors). Thus, the disclosure contemplates, in certain embodiments, the use of reprogramming factors that induce pluripotency (e.g., the reprogramming factor(s) are pluripotency factor(s), as well as reprogramming factors that alter cell fate but do not induce pluripotency. In certain embodiments, the pluripotency factors induce pluripotency. Cells produced by techniques employing one or more pluripotency factors include i) immortalized secondary somatic cells containing an inducible polycistronic exogenous nucleic acid encoding pluripotency factor(s); ii) somatic cells resulting from the expression of the pluripotency factor(s) in the immortalized secondary somatic cells (e.g., secondary iPSCs, secondary iMPCs); and iii) cells derived therefrom (e.g., tertiary somatic cells). It is contemplated that the capacity of a pluripotency factor to alter cell fate without inducing pluripotency may, in certain embodiments, be dependent in part by its level of expression. Thus, in certain embodiments, expression of an effective amount of pluripotency factor(s) is induced.

It is noted that suitable reprogramming factor(s) may be a single factor or a combination of more than one factor. In certain embodiments, the reprogramming factor(s) is a pluripotency factor(s) and is a combination of four factors.

In certain embodiments, the reprogramming factors are selected for their ability to induce de-differentiation, without producing a pluripotent cell or passing through a pluripotent state. Cells produced by techniques employing one or more reprogramming factors that induce de-differentiation without causing the cell to pass through pluripotency include i) immortalized secondary somatic cells containing an inducible polycistronic exogenous nucleic acid encoding a reprogramming factor(s); ii) somatic cells resulting from the expression of a reprogramming factor(s) in the immortalized secondary somatic cells (e.g. secondary iMPCs); and iii) cells derived therefrom (e.g., tertiary somatic cells).

In another aspect, the invention provides for a cell that i) contains an inducible first polycistronic exogenous nucleic acid encoding one or more pluripotency factors operably linked to a first regulatory sequence and ii) expresses a second exogenous nucleic acid encoding an immortalizing factor operably linked to a second regulatory sequence. In certain embodiments, the cell does not express the pluripotency factor(s) encoded in the inducible first polycistronic exogenous nucleic acid (i.e., an immortalized secondary somatic cell).

The invention provides for a cell produced by inducing expression in an immortalized secondary somatic cell as described above of the pluripotency factors encoded in the inducible first polycistronic exogenous nucleic acid (i.e., to produce a secondary iPSC or a secondary iMPC) and cells derived therefrom (e.g., tertiary somatic cells).

In certain embodiments, the expression of a reprogramming factor(s) precedes the expression of or other exposure to an additional factor(s), such as a factor that promotes differentiation (e.g., a differentiating factor(s)). In certain embodiments, the expression of a reprogramming factor(s) is concurrent with the expression of or exposure to an additional factor(s), such as a differentiating factor(s). It is contemplated that exposure to an additional factor(s), such as a differentiating factor, can be achieved by endogenous generation within the cell (e.g., via gene expression) or by exogenous treatment of the cell (e.g., adding the factor(s) to the culture medium). In certain embodiments, the cell is a human or a mouse cell and/or a somatic cell such as a fibroblast, keratinocyte, or adult stem cell. Exemplary adult stem cells include hematopoietic stem cells, neural stem cells, and mesenchymal stem cells.

In certain embodiments, the pluripotency factors are selected from Oct4, KLF4, Myc, and Sox2, and combinations thereof.

In certain embodiments, the immortalizing factor is (preferably) hTERT.

In one aspect, the invention provides a method of producing an immortalized secondary somatic cell, comprising: i) introducing into an initial cell an inducible first polycistronic exogenous nucleic acid encoding reprogramming factors operably linked to a first regulatory sequence; ii) inducing expression of the reprogramming factors; and iii) introducing into the secondary cell a second exogenous nucleic acid encoding an immortalizing factor operably linked to a second regulatory sequence, whereby the secondary cell expresses the immortalizing factor.

In certain embodiments, the method further comprises inducing expression of the reprogramming factors in an immortalized secondary somatic cell to effect direct reprogramming of that cell into a somatic cell of a second cell type, via a de-differentiated intermediate, such as a secondary iMPC, in the absence of undergoing a pluripotency stage (e. g., tertiary somatic cells).

In another aspect, the invention provides a method of producing an immortalized secondary somatic cell, comprising: i) introducing into an initial cell an inducible first polycistronic exogenous nucleic acid encoding pluripotency factors operably linked to a first regulatory sequence; ii) inducing expression of the pluripotency factors; iii) exposing the cell that expresses the pluripotency factors to differentiation agents to produce a secondary (somatic) cell; and iv) introducing into the secondary (somatic) cell a second exogenous nucleic acid encoding an immortalizing factor operably linked to a second regulatory sequence, whereby the secondary cell expresses the immortalizing factor.

In certain embodiments, the method further comprises inducing expression of the pluripotency factors encoded in the inducible first polycistronic exogenous nucleic acid in the secondary cell that expresses the immortalizing factors (e. g., to generate secondary iPSCs or secondary iMPCs), and may even include differentiating the secondary iPSCs or secondary iMPCs to generate tertiary somatic cells by exposing the secondary iPSCs to or secondary iMPCs differentiation agents.

In certain embodiments, the initial cell is a human or mouse cell, and/or is a somatic cell such as a fibroblast, keratinocyte, or adult stem cell. Exemplary types of adult stem cells include hematopoietic stem cells, neural stem cells, and mesenchymal stem cells. In certain embodiments, the initial cell is obtained from a subject. In certain embodiments, the initial cell or cell derived therefrom contains a third exogenous nucleic acid encoding a wild-type or mutant gene useful for modeling a condition in health or disease associated with that gene.

In certain embodiments, the pluripotency factors are selected from Oct4, KLF4, Myc, and Sox2, and combinations thereof, preferably a combination of all four.

In certain embodiments, the immortalizing factor is (preferably) hTERT.

The present invention further provides methods for identifying agents that affect nuclear reprogramming, and/or cellular differentiation, proliferation, viability, or metabolism, as well as for administering cells as described herein to a subject, e.g., to treat a patient who would benefit from receiving the cells.

The cells as described herein can be used to identify an agent that affects nuclear reprogramming, e.g., by exposing a cell (e.g., an immortalized secondary somatic cell or tertiary somatic cell, as described herein) to the test agent, and detecting, identifying, and/or quantifying a change in nuclear reprogramming, wherein in a change in nuclear reprogramming relative to an untreated control cell indicates that the test agent affects nuclear reprogramming.

Similarly, the cells as described herein can be used to identify an agent that affects cellular differentiation, such as by exposing a cell of the invention (e.g., an immortalized secondary somatic cell, secondary iPSC, or tertiary somatic cell, as described herein) to the test agent, and detecting, identifying, and/or quantifying a change in cellular differentiation, wherein in a change in cellular differentiation relative to an untreated control cell indicates that the test agent affects cellular differentiation.

The cells as described herein can also be used to identify an agent that affects cellular proliferation, e.g., by exposing a cell of the invention (e.g., an immortalized secondary somatic cell, secondary iPSC, or tertiary somatic cell, as described herein) to the test agent, and detecting, identifying, and/or quantifying a change in cellular proliferation, wherein in a change in cellular proliferation relative to an untreated control cell indicates that the test agent affects cellular proliferation.

Analogously, the cells as described herein can be used to identify an agent that affects cellular viability, such as by exposing a cell of the invention (e.g., an immortalized secondary somatic cell, secondary iPSC, or tertiary somatic cell, as described herein) to the test agent, and detecting, identifying, and/or quantifying a change in cellular viability, wherein in a change in cellular viability relative to an untreated control cell indicates that the test agent affects cellular viability.

The cells as described herein can also be used to identify an agent that affects cellular metabolism, e.g., by exposing a cell of the invention (e.g., an immortalized secondary somatic cell, secondary iPSC, or tertiary somatic cell, as described herein) to the test agent, and detecting, identifying, and/or quantifying a change in cellular metabolism, wherein in a change in cellular metabolism relative to an untreated control cell indicates that the test agent affects cellular metabolism.

Cells as described herein can also be used to treat a subject in need of cellular therapy. For example, a cell that is a secondary iPSC or a secondary iMPC may be exposed to differentiation agents, and the resulting cell (e. g., a tertiary somatic cell) implanted into the subject. Such cells may be used to restore, augment, or provide a desired function in the subject.

### BRIEF DESCRIPTION OF THE FIGURES

FIG. 1 FIGS. 1A and 1B are flowcharts depicting a general technique for engineering cells according to the invention.

FIG. 2 FIGS. 2A and 2B are schematic representations of a method for producing the cells of the disclosure, such as immortalized secondary somatic cells and secondary iPSCs. FIG. 2C is a schematic representation of elements contained in viral vectors, in this case lentiviral vectors, used for reprogramming and immortalization. The cells depicted in FIG. 2B are as follows: (i) hiBJ, which refers to a primary human BJ (hBJ) neonatal foreskin fibroblast that carries the inducible polycistronic exogenous nucleic acid encoding the reprogramming factor(s); (ii) hiF, which refers to a secondary human fibroblast that carries the inducible polycistronic exogenous nucleic acid encoding the reprogramming factor(s) (i.e., a secondary somatic cell shown in FIG. 1); (iii) hiF-T, which refers to a secondary human fibroblast that carries the inducible polycistronic exogenous nucleic acid encoding a reprogramming factor(s) and that expresses an immortalization factor encoded on a second exogenous nucleic acid (i.e., an immortalized secondary somatic cell shown in FIG. 1); (iv) hIPSC-T, which refers to a human induced pluripotent stem cell that carries the inducible polycistronic exogenous nucleic acid encoding the reprogramming factor(s) and the exogenous nucleic acid encoding the immortalization factor (e.g., a secondary iPSC shown in FIG. 1); and (v) hIPSC, which refers to a human induced pluripotent stem cell generated, e.g., from reprogramming a hiBJ cell (i.e., a primary iPSC shown in FIG. 1) or from a hiF cell. In the example set forth schematically in FIG. 2C, the reprogramming factors are pluripotency factors, and induction of the pluripotency factors and culture of the cells following such induction produce iPSCs. However, use of a reprogramming factor(s) that dedifferentiates or otherwise alters the fate of the somatic cell, such as to an iMPCs, is similarly contemplated.

FIG. 3 FIGS. 3A and 3B are a series of images showing the cells at various stages in the reprogramming and selection process leading to the generation of secondary somatic cells. iPSC positive for tra1-60 antigen (an art recognized marker of pluripotency) is shown, as well as immunofluorescence detecting the expression of the first transgene of the polycistron (i.e.: OCT4) upon DOX supplementation (+DOX). Secondary somatic cells grown in the absence of DOX (−DOX) do not express the first transgene of the polycistron.

FIG. 4 FIG. 4A is a schematic flowchart showing the selection of immortalized secondary somatic cells. FIG. 4B presents images showing immortalized secondary somatic cells (hiF-7/TERT-40) at passage 2 (left) and passage 15 (right).

FIG. 5 FIG. 5 is a series of images of reprogrammed immortalized secondary somatic cells (hiF-TERT) visualized by immunofluorescence staining for alkaline phosphatase (FIG. 5A), tra1-60 antigen (FIGS. 5B and 5D). A phase contrast image of FIG. 5D is shown in FIG. 5C.

FIG. 6 FIG. 6 is a series of images of primary BJ cells (hiBJ) and secondary hiF and hiF-T cells stained for alkaline phosphatase (AP) after 18 days of reprogramming.

FIG. 7 FIG. 7 is a series of plots showing the reprogramming efficiencies of primary human BJ (hiBJ) cells, secondary somatic (hiF) cells, and immortalized secondary somatic (hiF-T) cells. Reprogramming efficiencies were analyzed by (1) measuring the ratio of TRA-1-60+ colonies/starting cells at 18 days of reprogramming (FIG. 7A); (2) measuring the area of individual TRA-1-60+ cells after 18 days of reprogramming (FIG. 7B); and (3) determining the time of onset of TRA-1-60+ colonies during a 21 day reprogramming time course (FIG. 7C). The box plots show the first and the third quartiles, along with the medians. The ends of the whiskers represent the 2.5 and 97.5 percentiles. The line plots show medians and quartiles.

FIG. 8 FIG. 8 is a series of plots showing transgene expression of TERT (FIG. 8A), Oct4 (i.e. POUSFA) (FIG. 8B), KLF4 (FIG. 8C), MYC (FIG. 8D) and SOX2 (FIG. 8E) in BJ cells, immortalized secondary somatic cells (hiF-T-DOX; immortalized secondary somatic cells containing a polycistronic nucleic acid encoding pluripotency factor(s), but in which expression of the exogenously provided, polycistronic nucleic acid encoding pluripotency factors has not yet been induced), immortalized secondary somatic cells being reprogrammed (hiF-T+DOX; immortalized secondary somatic cells in which expression of the exogenously provided, polycistronic nucleic acid encoding pluripotency factors has been induced), hIPSCs derived from reprogrammed hiF-T cells (hIPSC-T; pluripotent cells produced by reprogramming hiF-T cells by inducing expression of the pluripotency factor(s) encoded by the polycistronic nucleic acid) and reference hESCs. The hESC values show the median values across 18 hESC lines (H1, H9 and HUES clones 1, 3, 8, 9, 13, 28, 44, 45, 48, 49, 53, 62, 63, 64, 65, 66).

FIG. 9 FIG. 9 is a series of images of hiF (secondary somatic cells) and hiF-T (immortalized secondary somatic cells) cells. FIG. 9A shows increased senescence (top) and reduced reprogramming capacity (bottom) of hiF cells after two weeks of culture. FIG. 9B shows hiF-T cells do not senescence (top) and retain their reprogramming capacity (bottom) even after three months in culture.

FIG. 10 FIG. 10 is a series of charts showing the expression of proliferation genes (FIG. 10A), stem cell genes (FIG. 10B) and senescence genes (FIG. 10C) in the secondary somatic (hiF) and immortalized secondary somatic (hiF-T) cells over time in hiF early (<P6), hiF mid (P6-P8), hiF late (>P8), hiF-T early (<P20), and hiF-T late (>P30) cells. (P=passage number) For each graph, the bars from left to right depict results for: BJ cells, hiF early cells, hiF mid cells, hiF late cells, hiF-T early cells, and hiF-T late cells.

FIG. 11 FIG. 11 is an image showing the hierarchical clustering of BJ, hiF and hiF-T cells according to expression levels of proliferation (left) or stem cell (right) genes.

FIG. 12 FIG. 12 is a series of images showing the karyotypes of a hiF-T cell at an early passage (P3) (FIG. 12A) and at late passage (P40) (FIG. 12B) and the karyotype of a derivative hIPSC-T cell (i.e., secondary iPSCs; iPSCs generated by reprogramming an immortalized, secondary somatic hiF-T cell by, for example, inducing expression of a polycistronic nucleic acid present in the hiF-T cells) at passage 13 (P13) (FIG. 12C).

FIG. 13 FIG. 13 is an image of a matrix showing differentially expressed genes between all possible pairings of the fibroblast populations, as determined by RNA-Seq. Early and late passages of hiF-T cells show the lowest number of differentially expressed genes (i.e., 59 genes circled in red).

FIG. 14 FIG. 14 depicts qRT-PCR expression levels of key early germ layer-specific genes in in vitro differentiated hIPSC-T cells. Briefly, hIPSC-T cells were differentiated in vitro to ectodermal (dEC), mesodermal (dME), or endodermal (dEN) cell types. Expression of key ectodermal genes is depicted in FIG. 14A, expression of key endodermal general is depicted in FIG. 14B, and expression of key mesodermal genes is depicted in FIG. 14C. For each graph, gene expression for each gene was evaluated and the relative expression level in undifferentiated hIPSC-T cells, differentiated ectoderm (dEC; ectodermal cells differentiated from the hIPSC-T cells), differentiated mesoderm (dME; mesodermal cells differentiated from the hIPSC-T cells), and differentiated endoderm (dEN; endodermal cells differentiated from the hIPSC-T cells) is shown as four bars for each gene evaluated. For each gene, four bars are presented depicting relative gene expression in, from left to right, undifferentiated hIPSC-T cells (blue bars), dEC (red bars), dME (orange bars) and dEN (yellow bars). Values are reported as fold change relative to undifferentiated hIPSC-T cells.

FIG. 15 FIG. 15 is a plot showing the scorecard differentiation scores for hIPSC-T cells directed to differentiate into ectodermal (dEC), mesodermal (dME) and endodermal (dEN) cells (solid bars; red bars, when viewed in color) on top of box plots showing the score distributions of 6 pluripotent cells lines that have been shown to generate all the three germ layers in vivo using teratoma assays.

FIG. 16 FIG. 16 is a series of plots showing the results of an RNAi screen performed using hIF-T cells to identify reprogramming regulators. FIG. 16A is a plot comparing selected reprogramming efficiencies in shRNA-perturbed hIF-T cells at day 15 in a pooled screening format (Y axis-enrichment of shRNA sequence reads from TRA-1-60+ cells versus cells prior to induction of reprogramming) versus an arrayed format (X axis-number of TRA-1-60+ colonies). Novel candidate regulators C1-C7 are indicated in the plot. LSD1, previously identified as a candidate regulator of reprogramming, was also identified in this screen. FIG. 16B is a plot showing the reprogramming efficiency (number of TRA-1-60+ colonies) upon shRNA-mediated perturbation of candidate regulators C1-C7 and LSD1 (upper panel) and the corresponding change in mRNA expression levels in hIF-T cells relative to the effect of a control shRNA targeting the luciferase (LUC) mRNA (lower panel). Three distinct hairpins were tested for each gene and one representative TRA-1-60 staining is displayed above each set.

FIG. 17 FIG. 17 is a series of images showing hBJ cells at various stages in the reprogramming and selection process leading to the generation of muscle cells.

### DETAILED DESCRIPTION

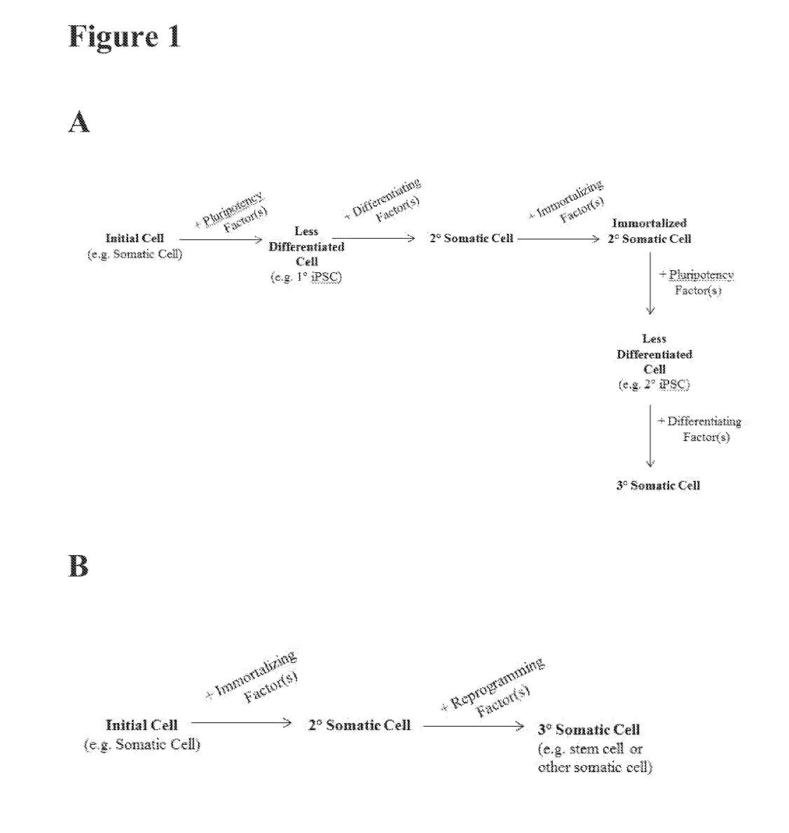

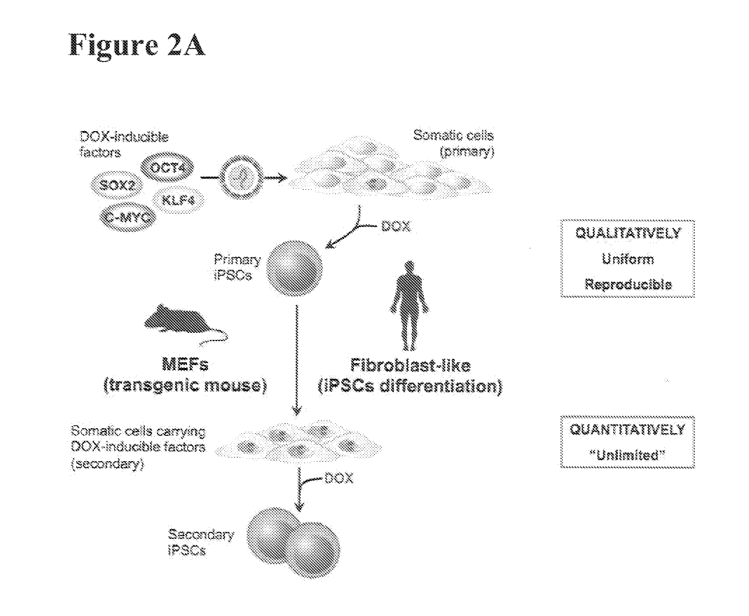

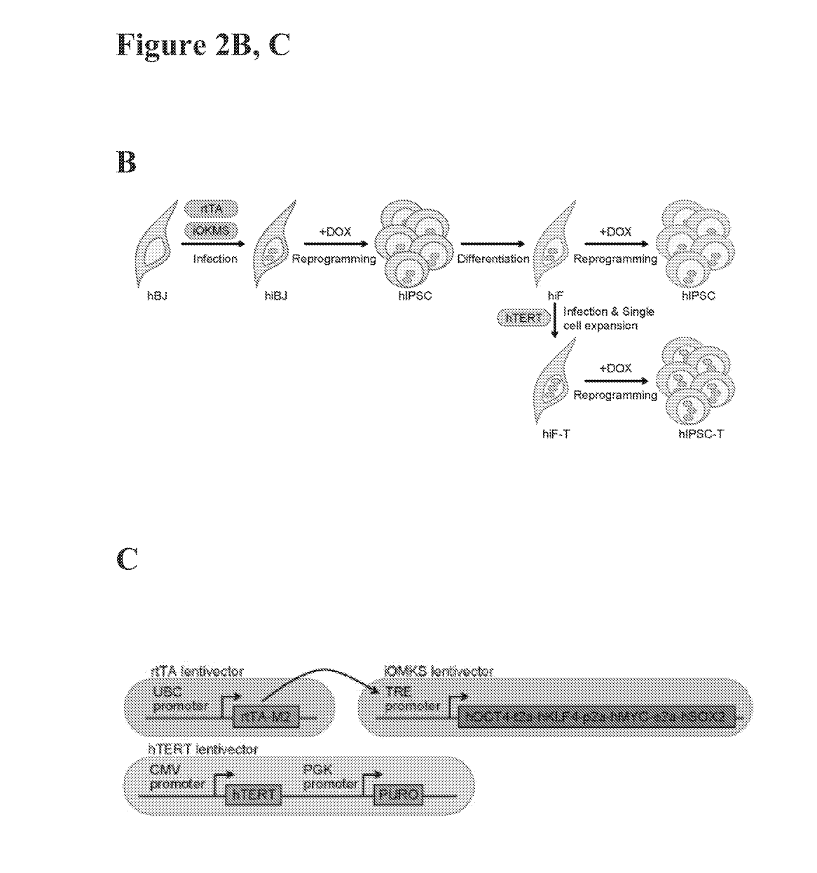

The present invention provides techniques for engineering cells useful for a variety of research and therapeutic purposes. The pathway for engineering these cells (shown in FIGS. 1A, 1B, and 2A-C) provides a variety of distinct cell types, including i) immortalized secondary somatic cells containing an inducible polycistronic exogenous nucleic acid encoding one or more reprogramming factors derived either directly from an initial cell (via a de-differentiated intermediate that does not pass through pluripotency, such as a primary iMPC), or indirectly via a de-differentiated cell that passes through pluripotency (such as a primary induced pluripotent stem cell (iPSC), ii) a secondary de-differentiated cell (such as a secondary iPSC or secondary iMPC) induced to express said pluripotency factors, and iii) tertiary somatic cells derived from immortalized secondary somatic cells either directly (e.g., by inducing expression of the reprogramming factors in an immortalized secondary somatic cell via a de-differentiated intermediate that does not without pass through a discrete pluripotency stage, (such as a secondary iMPC), or indirectly via a secondary de-differentiated cell that passes through a discrete pluripotency stage (such as a secondary iPSC). The present invention relates to each individual step depicted in FIGS. 1 and 2 (and all combinations of successive steps, as well as the complete series of steps), and each individual cell type depicted in FIGS. 1 and 2. The present invention further provides methods for employing such cells in assays to identify agents that affect nuclear reprogramming, and/or cellular differentiation, proliferation, viability, or metabolism, as well as for methods of administering cells as described herein to a subject, e.g., to treat a patient who would benefit from receiving the cells.

The present invention provides cells that are particularly well suited, for example, for screening and research applications. By way of example, in certain embodiments, a culture of immortalized secondary somatic cells provided herein and/or produced by the methods provided herein, are more uniform to one another, e.g. in kinetics of transcriptional activity and in size and, for a given population derived from a given starting cell, are clonal. Thus, in certain embodiments, a culture of immortalized secondary somatic cells is a substantially homogenous culture of cells, from which substantially homogenous culture of secondary iPSCs, secondary iMPCs, and tertiary somatic cells can be derived. Such uniform cultures, such as substantially homogenous cultures of immortalized secondary somatic cells are well suited for screening assays and research applications because significant heterogeneity in a culture system or cell population can obscure results and lead to false positives and false negatives. Such cells are useful in assays and/or screening and/or research for understanding reprogramming, cell fate, and development, and can be used as single cells or as cultures of cells. More uniform cultures are well suited as a starting point for single cell analysis and single cell screening assays for the same reasons. The immortalized secondary somatic cells provided herein and/or produced by the methods provided herein, also have robust proliferative capacities that enable the generation of large quantities of uniform cells useful for large scale screening assays and research applications.

The invention provides a method of producing an immortalized secondary somatic cell, comprising: i) introducing into an initial cell an inducible first polycistronic exogenous nucleic acid encoding one or more reprogramming factors operably linked to a first regulatory sequence; ii) inducing expression of the reprogramming factors to produce a secondary cell; and iii) introducing into the secondary cell a second exogenous nucleic acid encoding an immortalizing factor operably linked to a second regulatory sequence, whereby the secondary cell expresses the immortalizing factor. The reprogramming factors can be any factor or combination of factors that reprograms the cell to resemble or become a different cell type of interest. Such factors and combinations of factors are well known in the art, and can be selected from one or more of the reprogramming factors disclosed herein, but any factors or combinations of factors that effectively reprogram a cell to provide a desired phenotype can be employed. In certain embodiments, the reprogramming factor(s) comprise pluripotency factors, and induction of expression of the pluripotency factors expressed from the exogenously provided polycistronic nucleic acid reprograms the cell to a pluripotent cell, e.g., an iPSC.

Alternatively, the invention provides a method of producing an immortalized secondary somatic cell, comprising: i) introducing into an initial cell an inducible first polycistronic exogenous nucleic acid encoding pluripotency factors operably linked to a first regulatory sequence; ii) inducing expression of the pluripotency factors encoded in the inducible first polycistronic exogenous nucleic acid (to generate e.g., primary iMPCs or primary iPSCs); iii) exposing the cell that expresses the pluripotency factors to differentiation agents to produce a secondary cell; and iv) introducing into the secondary cell a second exogenous nucleic acid encoding an immortalizing factor operably linked to a second regulatory sequence, whereby the secondary cell expresses the immortalizing factor. The pluripotency factors can be any factor or combination of factors that reprograms the cell to resemble or become a de-differentiated cell, such as an embryonic-like stem cell (or iPSC) or a multipotent progenitor cell. In certain embodiments, the method of producing an immortalized secondary somatic cell further comprises selection and clonal expansion of an individual cell generated in step ii and step iv. In certain embodiments, the secondary cell is a fibroblast and differentiation agents comprise, for example, culturing in fibroblast media under know fibroblast differentiation conditions.

The invention provides a method of producing a secondary de-differentiated cell (e.g., secondary iPSC, secondary iMPC), comprising inducing expression of the pluripotency factor(s) encoded in the inducible first polycistronic exogenous nucleic acid. The pluripotency factors can be any factor or combination of factors that reprograms the cell to resemble or become a de-differentiated cell, such as an embryonic-like stem cell (or iPSC) or a multipotent progenitor cell.

Alternatively, the invention further provides a method of producing a secondary de-differentiated cell (e.g., secondary iMPC), comprising i) inducing expression of the reprogramming factor(s) encoded in the inducible first polycistronic exogenous nucleic acid. The reprogramming factors can be any factor or combination of factors that reprograms the cell to resemble or become a de-differentiated cell, such as a multipotent progenitor cell.

The invention further provides a method of producing tertiary cells, comprising inducing the secondary de-differentiated cell to differentiate, for example by exposing the secondary de-differentiated cell to a differentiating factor(s). The differentiating factors can be any factor or combination of factors that directly or indirectly induces the de-differentiated cell to differentiate.

The invention also provides a method of producing a tertiary cell, comprising i) introducing into an initial cell a first exogenous nucleic acid encoding an immortalizing factor operably linked to a first regulatory sequence, whereby the initial cell expresses the immortalizing factor; ii) introducing into the initial cell that expresses the immortalizing factor (i.e., second somatic cell) an inducible second polycistronic exogenous nucleic acid encoding one or more reprogramming factors operably linked to a second regulatory sequence; and iii) inducing expression of the reprogramming factors to produce a tertiary cell. The reprogramming factors can be any factor or combination of factors that reprograms the initial cell to resemble or become a different cell type of interest. Such factors and combinations of factors are well known in the art, and can be selected from one or more of the reprogramming factors disclosed herein, but any factors or combinations of factors that effectively reprogram a cell to provide a desired phenotype can be employed.

In certain embodiments, the method provides for single-cell expansion of individual clones to ensure genetic uniformity across the entire proliferated population. In certain preferred embodiments, individual reprogrammed cells are isolated prior to expansion and subsequent manipulations. In certain preferred embodiments, individual immortalized cells are isolated prior to expansion and subsequent manipulations.

In certain embodiments, the initial cell is a human or mouse cell. In certain embodiments, the initial cell is a human cell. In certain embodiments, the initial cell is a somatic cell, such as a fibroblast, keratinocyte, neuron, muscle cell or other cell derived from any of the three germ cells layers (i.e.: mesoderm, ectoderm and endoderm). In certain embodiments, the initial cell is a post-natal cell, such as a post-natal somatic cell. In certain embodiments, the cell is an adult cell. In certain embodiments, the initial cell is an adult stem cell. Exemplary adult stem cells include hematopoietic stem cells, neural stem cells, and mesenchymal stem cells.

In certain embodiments, the secondary somatic cell is a human or mouse cell. In certain embodiments, the secondary somatic cell is a human cell. In certain embodiments, the secondary somatic cell is somatic cell such as a fibroblast, keratinocyte, neuron, muscle cell or other cell derived from any of the three germ cells layers (i.e.: mesoderm, ectoderm and endoderm). In certain embodiments, the secondary somatic cell is a post-natal cell, such as a post-natal somatic cell. In certain embodiments, the cell is an adult cell. In some embodiments, the secondary cell is an adult stem cell. Exemplary adult stem cells include hematopoietic stem cells, neural stem cells, and mesenchymal stem cells.

Consolidating multiple pluripotency or reprogramming factors into a single polycistronic construct limits genetic variability when transfecting initial cells with the construct by minimizing the number of integration sites into the genome of the initial cell, as compared to methods that use multiple constructs that integrate at multiple sites in the genome. The transduced initial cells represent a more genetically homogeneous starting material from which homogenous iPSCs, secondary somatic cells, etc. can be generated in a highly reproducible manner for use as described herein. Furthermore, use of the inducible polycistronic construct encoding the pluripotency or reprogramming factors ensures that the pluripotency or reprogramming factors are expressed in a desired stoichiometry relative to each other in transfected initial cells for efficient reprogramming and production of iPSCs.

Accordingly, the invention provides a cell that i) contains an inducible first polycistronic exogenous nucleic acid encoding one or more reprogramming factors (e.g., pluripotency factors) operably linked to a first regulatory sequence and ii) expresses a second exogenous nucleic acid encoding an immortalizing factor operably linked to a second regulatory sequence. In certain embodiments, the cell does not express the reprogramming or pluripotency factors encoded in the inducible first polycistronic exogenous nucleic acid (i.e., an immortalized secondary somatic cell).

In preferred embodiments, the immortalized secondary somatic cells of the disclosure maintain high reprogramming potential and at least certain genetic and/or epigenetic characteristics of primary cells, allowing the immortalized secondary somatic cells and their progeny to be used in place of the corresponding primary cells in situations where direct use of primary cells would be impractical or otherwise ineffective.

The immortalized secondary somatic (hiF-T) cells of the disclosure have herein been shown to be capable of generating secondary iPSC (hIPSC-T) that retain the capability to differentiate into cells of each of the three germ layers (e.g., cells having a differentiation potential similar to that of embryonic and induced pluripotent stem cells of the same species as the iPSC). In other words, immortalized secondary somatic cells can be reprogrammed to, for example, iPSCs. Therefore, in certain embodiments, the immortalized secondary somatic (hiF-T) cell is capable of being reprogrammed into a cell that can then be differentiated into an ectodermal, endodermal, or mesodermal cell. It is contemplated that ability of the secondary iPSCs to acquire a naive pluripotent state reminiscent of hESCs, as shown herein, underlies the capability of these cells to differentiate into a wide variety of tertiary somatic cell types. Accordingly, in certain embodiments, the reprogrammed immortalized secondary somatic (hiF-T) cell (i.e., secondary iPSC (hIPSC-T)) is capable of being differentiated into an ectodermal cell, an endodermal cell, or a mesodermal cell (e.g., the cells are capable of being differentiated to cell types of all three germ layers).

In certain embodiments, the reprogrammed immortalized secondary somatic (hiF-T) cell (i.e., secondary iPSC (hIPSC-T)) is capable of being differentiated into a cell characterized by the expression of an ectodermal gene comprising one or more of: CDH9, DMBX1, DRD4, LMX1A, MYO3B, NOS2, NR2F1, NR2F2, OLFM3, PAX3, PAX6, POU4F1, TRPM8, WNT1, and ZBTB16, either alone or in combination with one, two, three, or more ectodermal genes. In certain embodiments, the cell expresses a combination of any of the foregoing ectodermal genes (e.g., 2, 3, 4, 5, 6, 7, 8, 9, 10, 11, 12, 13, 14, 15), optionally, in combination with one or more other genes. In certain embodiments, the reprogrammed immortalized secondary somatic (hiF-T) cell (i.e., secondary iPSC (hIPSC-T)) is capable of being differentiated into a cell characterized by the expression of an endodermal gene comprising one or more of: EOMES, FOXA1, FOXA2, FOXP2, GATA4, GATA6, HHEX, HNF1B, HNF4A, KLF5, LEFTY1, LEFTY2, NODAL, RXRG, and SOX17, either alone or in combination with one, two, three, or more endodermal genes. In certain embodiments, the cell expresses a combination of any of the foregoing endodermal genes (e.g., 2, 3, 4, 5, 6, 7, 8, 9, 10, 11, 12, 13, 14, 15), optionally, in combination with one or more other genes. In certain embodiments, the reprogrammed immortalized secondary somatic (hiF-T) cell (i.e., secondary iPSC (hIPSC-T)) is capable of being differentiating into a cell characterized by the expression of a mesodermal gene comprising one or more of: ABCA4, BMP10, CDX2, ESM1, FOXF1, HAND1, HAND2, HEY1, HOPX, NKX2-5, ODAM, PLVAP, RGS4, SNAI2, and TBX3, either alone or in combination with one, two, three, or more mesodermal genes. In certain embodiments, the cell expresses a combination of any of the foregoing mesodermal genes (e.g., 2, 3, 4, 5, 6, 7, 8, 9, 10, 11, 12, 13, 14, 15), optionally, in combination with one or more other genes.

Furthermore, a tertiary somatic cell derived from a reprogrammed immortalized secondary somatic (hiF-T) cell can be an endodermal cell, an ectodermal cell, or a mesodermal cell. Accordingly, in certain embodiments, the tertiary somatic cell derived from the reprogrammed immortalized secondary somatic (hiF-T) cell is characterized by the expression of an endodermal gene comprising one or more of: EOMES, FOXA1, FOXA2, FOXP2, GATA4, GATA6, HHEX, HNF1B, HNF4A, KLF5, LEFTY1, LEFTY2, NODAL, RXRG, and SOX17, either alone or in combination with one, two, three, or more endodermal genes. In certain embodiments, the tertiary somatic cell derived from the reprogrammed immortalized secondary somatic (hiF-T) cell is characterized by the expression of an ectodermal gene comprising one or more of: CD19, DMBX1, DRD4, LMX1A, MYO3B, NOS2, NR2F1, NR2F2, OLFM3, PAX3, PAX6, POU4F1, TRPM8, WNT1, and ZBTB16, either alone or in combination with one, two, three, or more ectodermal genes. In certain embodiments, the tertiary somatic cell derived from the reprogrammed immortalized secondary somatic (hiF-T) cell is characterized by the expression of a mesodermal gene comprising one or more of: ABCA4, BMP10, CDX2, ESM1, FOXF1, HAND1, HAND2, HEY1, HOPX, NKX2-5, ODAM, PLVAP, RGS4, SNAI2, and TBX3, either alone or in combination with one, two, three, or more mesodermal genes.

The reprogramming process is characterized by a continuous trajectory of transcriptional changes beginning at the immortalized secondary somatic (hiF-T) cells and ending with the fully established secondary iPSC (hIPSC-T), as shown herein. The immortalized secondary somatic cells disclosed herein show striking homogeneous kinetics of transcriptional activity characterized by several transient waves of gene regulation (reflecting various stages of differentiation or de-differentiation) resulting in the differential expression of a multitude of genes.

In certain embodiments, an immortalized secondary somatic cell disclosed herein can be characterized by the differential expression (e.g., up-regulation or down-regulation relative to a reference hESC) of one or more markers comprising: CDK1, AURKA, MYBL2, PGF, DPPA4, DPPA3, LIN28A, LIN28B, FILIP1L, IGF2, IGFBP2, SSEA-3, TRA-1-60, TRA2A, SNAI2, H19, LEFTY2, UTF1, OTX2, miR-10, miR-221, miR-371, miR-302, miR-25, miR-515, BRDT, DND1, ELF5, LOXHD1, TDRD12, DNMT3B, DNMT3L SYC P1, EZHI, LSD1, KTI12, LBR, NAP1L3, PHF16, RSF1, SHPRH, ROCK, ALDH1A1, ALDH1A2, HNF4A, APOE, CCND2, CDH1, CDH9, TERT, OCT4, KLF4, KLF5, MYC, SOX4, SOX17, DMBX1, DRD4, LMX1A, MYO3B, NOS2, NR2F1, NR2F2, OLFM3, PAX3, PAX6, POU4F1, TRPM8, WNT1, ZBTB16, EOMES, FOXA1, FOXA2, FOXCA2, FOXF1, FOXD3, FOXP2, GATA4, GATA6, HHEX, LEFTY1, LEFTY2, NODAL, RXRG, ABCA4, BMP10, CDX2, ESM1, HAND2, HEY1, HOPX, NKX2-5, ODAM, PLVAP, RGS4, TBX3, COL1A2, ANPEP (CD13), CD44, HOXA5/6/7/9/10/11, HOXD1/8/9/10/11/13, MEIS1/2, SIX1, AFP, GSN, CAVI, DCN, FOSL1, SLC6A3, FRAT2, HPGD, NEFL, WNT3/9A/10B, ACTA1, GDF3, NANOG, TFCP2L1, ALPPL2, CNNA1, DPPA2/3/4/5, FGFR4, FGF4, NLRP7, NLRP12, ZFP42, ZFP57, KIT, SALL2, SALL4, ALPL, TDGF1, ADCY2, B3GAT1, EMX1, GABRB3, SOX3, NLPP7, NLRP7, DPPA3, DNMT3L, CST1, DPPA2, CR1L, ALPPL2, ERVH48-1, DPPA5, TCL1B, ZFP57, CER1, OLAH, FGF17, CCNA1, FGF4, ZYG11A, RAB17, VRTN, PDZD4, ADCY2, GABRB3, OTX2, TSTD1, CD74, SALL1, CACNG7, KCNQ2, SCG3, CHGA, HEPH, OLFM1, PPP2R2B, SHANK2, CPZ, TRDN, VAT1L, CRIP3, TCEAL2, and PTGIS. In certain embodiments, an immortalized secondary somatic cell can be characterized by the differential expression of at least 2, 3, 4, 5, 6, 7, 8, 9, 10, 15, 20, or greater than 20 of the foregoing.

Characterization of cells disclosed herein based on any one or more of the marker genes discussed above may be determined at the level of the protein or RNA. For example, expression of a polypeptide encoded by the marker gene can be quantitatively determined using an antibody specific for the protein encoded by the marker gene in various assays such as enzyme immunoassays, radioimmunoassays, flow cytometry, and solid phase enzyme immunoassays. Alternatively, RNA expression of marker genes can be determined using for example a method comprising amplifying cDNA obtained by reverse transcription of mRNA from the cell and performing quantitative RT-PCR using marker gene(s) as the target; a northern blotting method comprising directly determining the mRNA level using a probe; a method of analyzing the expression level of mRNA using a DNA microarray carrying marker gene(s), and a method comprising extracting total RNA according to known methods and using, for example, an RNA-Seq Illumina library. In certain embodiments, TRA-1-60 expression refers to protein expression, and TRA-1-60+ cells are cells expressing TRA-1-60 antigen.

The immortalized secondary somatic (hiF-T) cells of the disclosure are uniform and acquire a fully somatic expression profile. The immortalized secondary somatic (hiF-T) cells also show high levels of genomic stability and transcriptional stability over prolonged periods of culture. It is contemplated that the prevention of senescence by immortalization of the secondary somatic cells enables the cells to fully silence stem-cell related genes and differentiate into a population of homogeneous fully differentiated somatic cells.

In certain embodiments, the immortalized secondary somatic cell of the disclosure is characterized by the differential expression (e.g., up-regulation or down-regulation relative to a reference cell) of one, two, three or more genes selected from the following: CD-13, SNAI2, miR-221, miR-10, TRA-1-60, SSEA-3, IGF2, H19, LEFTY2, UTF1, DPPA3, LIN28A, OCT4 and OTX2. In certain embodiments, the immortalized secondary somatic cell of the disclosure is characterized by the upregulation (e.g., presence or increased expression relative to a reference hESC) of one, two, three or more genes selected from the following: CD-13, SNAI2, miR-221 and miR-10. In certain embodiments, the immortalized secondary somatic cell of the disclosure is characterized by the down-regulation (i.e., absence or decreased expression relative to a reference hESC) of one, two, three or more genes selected from the following: TRA-1-60, SSEA-3, IGF2, H19, LEFTY2, UTF1, DPPA3, LIN28A, OCT4 and OTX2. In certain embodiments, the immortalized secondary somatic cell of the disclosure is characterized by the upregulation (e.g., presence or increased expression relative to a reference hESC) of CD-13, SNAI2, miR-221 and miR-10; and the down-regulation (i.e., absence or decreased expression relative to a reference hESC) of TRA-1-60, SSEA-3, IGF2, H19, LEFTY2, UTF1, DPPA3, LIN28A, OCT4 and OTX2.

In certain embodiments, the secondary iPSC of the disclosure is characterized by the differential expression (e.g., up-regulation or down-regulation relative to a reference cell) of one, two, three or more genes selected from the following: TRA-1-60, LIN28A, OTX2, OCT4, KLF4, MYC, SOX2, miR-25, mIR-302, CD-13, UTF1 and DPPA3. In certain embodiments, the secondary iPSC of the disclosure is characterized by the upregulation (e.g., presence or increased expression relative to a reference cell) of one, two, three or more genes selected from the following: TRA-1-60, LIN28A, OTX2, OCT4, KLF4, MYC, SOX2, miR-25, and mIR-302. In certain embodiments, the secondary iPSC of the disclosure is characterized by the down-regulation (i.e., absence or decreased expression relative to a reference cell) of one, two, three of the following genes: CD-13, UTF1, and DPPA3. In certain embodiments, the secondary iPSC of the disclosure is characterized by the upregulation (e.g., presence or increased expression relative to a reference cell) of TRA-1-60, LIN28A, OTX2, OCT4, KLF4, MYC, SOX2, miR-25, and mIR-302; and the down-regulation (i.e., absence or decreased expression relative to a reference cell) of CD-13, UTF1, and DPPA3.

In certain embodiments, the cells of the disclosure can be identified based on their distinctive chromatin state and/or DNA methylation patterns.

Immortalization of secondary somatic cells coupled with suitable growth conditions can allow expansion of these immortalized secondary somatic cells for at least 10, 15, 20, 25, 30, 35, or even 40 or more population doublings, providing a vast source of highly uniform (e.g., genetically and phenotypically uniform) cells and cells derived therefrom, which can be highly advantageous in high-throughput screening and other applications where uniformity across a large cell population is beneficial. Accordingly, the invention contemplates cells that are capable of proliferating through at least 10, 15, 20, 25, 30, 35, or even 40 or more population doublings, e.g., without significant loss of the desired phenotype of the original immortalized cell, and without a significant number of cells in the population senescing (e.g., less than 30%, 25%, 20%, 15%, 10%, or even less than 5% senescent cells in the population. Similarly, the invention provides large populations of highly uniform cells (e.g., at least 4 million, 8 million, 16 million, 32 million, 64 million, or even 128 million or more substantially uniform cells), and the methods described herein can include steps of proliferating the immortalized cells through at least 10, 15, 20, 25, 30, 35, or even 40 or more population doublings, e.g., prior to inducing expression of reprogramming or pluripotency factors or otherwise using the proliferated immortalized cells in any of the methods and procedures discussed herein.

In preferred embodiments of the cells and methods described herein, expression of the immortalizing factor is capable of being terminated. In some embodiments, immortalization factor may be constitutively expressed after introduction into the cell, but is silenced as a result of epigenetic changes that occur during reprogramming of the cell. In other embodiments, the expression of the immortalization factor may be regulated by employing a conditional promoter that controls its expression, allowing its expression to be selectively activated and deactivated. Because immortalization tends to limit the capacity of the cell to differentiate, the conditional expression of the immortalizing factor facilitates the generation of fully differentiated cells, which can be broadly used for a variety of purposes. Inducing expression of reprogramming factors in an immortalized secondary somatic cell (as described above) of a first cell type can directly reprogram that cell into a tertiary somatic cell of a second cell type, that passes through an induced multipotent progenitor stage without passing through a pluripotency stage. Accordingly, the invention contemplates cells in which both the reprogramming factor(s) and the immortalizing factor are expressed, as well as tertiary somatic cells in which the reprogramming factor(s) is/are not expressed and the immortalizing factor is expressed.

Similarly, inducing expression of pluripotency factors encoded in the inducible first polycistronic exogenous nucleic acid in the immortalized secondary somatic cell described above generates a de-differentiated (or less differentiated) cell such as a secondary iPSC or secondary iMPC. Such secondary iPSCs are directly comparable to those obtained from established primary reprogramming strategies as shown by extensive profiling of isolated intermediate populations using high-throughput strategies, including RNA-seq and ChIP-seq.

The de-differentiated cells such as secondary iPSCs or secondary iMPCs described herein are capable of being differentiated into a wide spectrum of tertiary somatic cells that resemble characteristics of primary cells. Suitable differentiation protocols are well known in the art, and any differentiation protocol that can differentiate an iPSC or iMPC to a cell that is or resembles a desired type of somatic cell may be employed to differentiate the secondary iPSCs or secondary iMPCs of the invention.

In certain embodiments, the tertiary somatic cell is a human or mouse cell. In certain embodiments, the tertiary somatic cell is a human cell. In certain embodiments, the tertiary somatic cell is somatic cell such as a fibroblast, keratinocyte, neuron, muscle cell or other cell derived from any of the three germ cells layers (i.e.: mesoderm, ectoderm and endoderm). In certain embodiments, the tertiary somatic cell is a post-natal cell, such as a post-natal somatic cell. In certain embodiments, the tertiary somatic cell is an adult cell. In some embodiments, the tertiary cell is an adult stem cell. Exemplary adult stem cells include hematopoietic stem cells, neural stem cells, and mesenchymal stem cells.

In certain embodiments, the reprogramming factors are selected from an Oct family gene (such as Oct4), a Klf family gene (such as Klf1, Klf2, Klf4 and Klf5), a Sox family gene (such as Sox1, Sox2, Sox3, Sox15, Sox17, and Sox18), a Myc family gene (such as c-Myc, L-Myc, and N-Myc), a Lin family gene (such as Lin28 and Lin28b), a methyl-CpG-binding protein family gene (such as MBD2), a Ascl family gene, a Neurogenin family gene, a NeuroD family gene, a Brn family gene, a Myt family gene, an Olig family gene, a Zic family gene, Nanog, MyoD, Esrrb, Esrrg, Musashil, GATA4, MEF2C, TBX5 (GMT) and Glis1, or a combination of two, three, four, or more genes thereof. In certain embodiments, it may be preferable to omit Klf family genes depending on the situation. The foregoing genes, and additional members of the recited gene families, are known in the art and the sequence of suitable human or mouse orthologs is known and can be readily selected. See, for example: NM_203289, NM_002701, NM_006563, NM_016270, NM_004235, NM_001730, NM_005986, NM_003106.3, NM_005634, NM_006942.1, NM_006942.1, NM_022454.4, NM_018419.2, NM_002467, NM_001033081, NM_005378, NM_024674, NM_001004317, NM_024865, NM_003927.4, NM_004316.3, NM_006161.2, NM_024019.3, NM_011820.1, NM_006236.1, NM_002700.2, NM_138983.2, NM_005806.3, NM_003412.3, NM_007129.3, NM_003413.3, NM_002478.4, NM_004452.3, NM_001134285.2, NM_002442.3, NM_002052.3, NM_001131005.2, NM_000192.3, and NM_147193.2.

Gene sequences encoding reprogramming factors derived from mammals (e.g., humans, mice, rats, bovines, sheep, horses, and monkeys) are preferred for use in the present invention. In addition to wild-type sequences, mutant sequences whose translation products have several (e.g., 1 to 10, preferably 1 to 6, more preferably 1 to 4, more preferably 1 to 3, particularly preferably 1 or 2) amino acids substituted, inserted, and/or deleted, and possess a function similar to that of the wild type peptide, can also be utilized. In certain embodiments, Myc mutants, variants, homologs, or derivatives may be used, such as mutants that have reduced transformation of cells. Examples include LMYC (NM.sub.--001033081), MYC with 41 amino acids deleted at the N-terminus (dN2MYC), or MYC with mutation at amino acid position 136 (e.g., W136E). In certain embodiments, the wild type Myc gene encoding stable type mutant (T58A) may be used. Similarly, genes encoding variants of other reprogramming factors disclosed herein that preserve their desired functionality are known in the art and/or can be prepared by those of skill in the art. Genes that encode the reprogramming factors disclosed herein are described in further detail in WO2007/69666, WO2013/0065311, WO2013/0040302, WO2013/0022583, WO2012/0288936, and Fu J D et al., (2013) Stem Cell Report 1(3):235-247 of which the disclosures of reprogramming factors and their uses are herein incorporated by reference in their entirety. Those skilled in the art can select sequences that can suitably be used in the methods of the present invention as appropriate by referring to the aforementioned publications and other documents in the published literature.

Furthermore, in addition to the aforementioned genes, one or more genes encoding a factor selected from Fbx15, ERas, ECAT15-2, Tcl1, and beta-catenin may be combined, and/or one or more nucleic acids selected from ECAT1, Esg1, Dnmt3L, ECAT8, Gdf3, ECAT15-1, Fthl17, Sal14, Rex1, UTF1, Stella, Stat3, and Grb2 may also be combined. These combinations are specifically described in WO2007/69666, which is herein incorporated by reference in its entirety for the disclosures related to reprogramming factors. Other examples of genes that encode reprogramming factors are disclosed in Science, 318, pp. 1917-1920, 2007, WO2008/118820, and the like, which are herein incorporated by reference in their entirety for the disclosures related to reprogramming factors. Those skilled in the art are able to understand the diversity of combinations of nucleic acids that encode nuclear reprogramming factors and determine suitable combinations for a particular purpose. Furthermore, by utilizing a nuclear reprogramming factor screening method described in WO 2005/80598, appropriate combinations of genes other than the combinations described in WO2007/69666 and Science, 2007 (supra) can be utilized in the methods provided herein, of which the disclosures related to reprogramming factors and methods are herein incorporated by reference in their entirety.

In certain embodiments, the pluripotency factors are selected from the reprogramming factors disclosed above. In certain embodiments, the pluripotency factors are selected from Oct4, KLF4, Myc, Sox2, Lin28, and Nanog and combinations thereof. In certain preferred embodiments, the pluripotency factors are selected from Oct4, KLF4, Myc, and Sox2, and combinations thereof, preferably a combination of all four.

A nucleic acid encoding the reprogramming factor(s), such as a polycistronic nucleic acid encoding pluripotency factor(s), can be introduced by transfection or transduction into the somatic cells using an integrating or non-integrating vector. In certain embodiments, the vector is an integrating vector. In certain embodiments, the vector is selected from plasmids, viral vectors (such as retroviral vectors, lentiviral vectors, and adenoviral vectors), artificial chromosomes (such as human artificial chromosomes (HACs), yeast artificial chromosomes (YACs), and bacterial artificial chromosomes (BACs or PACs)), episomal vectors (Yu et al. (2009)) Science, 324, 797-801), transposones (e.g., piggyback transposone: K. Kaji et al. (2009) Nature, 458:771-775; and Woltjen et al. (2009) Nature, 458:766-770) and the like. Furthermore, the vector can be introduced into cells, such as somatic cells, using conventional methods, such as an electroporation method, a microinjection method, a calcium phosphate method, a viral infection method, a lipofection method, and a liposome method. In certain preferred embodiments, the vector encoding the reprogramming factor(s) is a lentiviral vector. In certain preferred embodiments, such a lentivirus is introduced into the somatic cells by viral infection. In certain embodiments, the reprogramming factor(s) comprises more than one factor. When more than one factor is delivered, the nucleic acid encoding the plurality of factors may be polycistronic. This facilitates expression of each of the plurality of factors in the same cells, at the same copy number, and with the same integration cite. However, a polycistronic nucleic acid is, in certain embodiments, not used. For example, when a single factor is delivered, a polycistronic nucleic acid is not needed. In certain embodiments, the nucleic acid is inducible, such that expression of the reprogramming factor is regulated and is only turned on in response to another agent (e.g., a drug that can be added to the culture media).

An immortalizing factor may also, in certain embodiments, be delivered using any of the foregoing vector and transfection/transformation/transduction systems.

A vector to be used herein can contain regulatory sequences such as a promoter, an enhancer, a ribosome binding sequence, a terminator, and a polyadenylation site so that a nuclear factor can be expressed. Furthermore, if necessary, such a vector can further contain a selection marker sequence such as a drug resistance gene (e.g., a kanamycin resistance gene, an ampicillin resistance gene, and a puromycin resistance gene), a thymidine kinase gene, and a diphtheria toxin gene, and a reporter gene sequence such as a green fluorescent protein (GFP), beta glucuronidase (GUS), and FLAG. Moreover, the above vector can further contain LoxP sequences at the 5′-end and 3′-end of a gene encoding a reprogramming factor or a gene encoding a reprogramming factor linked to a promoter, which allows it to cleave the gene after introduction into somatic cells as disclosed in WO 2013014929.

In addition to the one or more reprogramming factors, the reprogramming technique may include contacting the cell with one or more other reagents as disclosed in WO2013/0022583, and which is hereby incorporated by reference in its entirety for the disclosures related to additional agents used in reprogramming systems. For example, the reprogramming system may include one or more agents known in the art to promote cell reprogramming. Examples of agents known in the art to promote cell reprogramming include GSK-3 inhibitors (e.g. CHIR99021 and the like (see, e.g., Li, W. et al. (2009) Stem Cells, Epub Oct. 16 2009)); histone deacetylase (HDAC) inhibitors (e.g., those described in US20090191159, the disclosure of which is incorporated herein by reference); histone methyltransferase inhibitors (e.g. G9a histone methyltransferase inhibitors, e.g. BIX-01294, and the like (see, e.g. Shi, Y et al. (2008) Cell Stem Cells 3(5):568-574)); agonists of the dihydropyridine receptor (e.g. BayK8644, and the like (see, e.g., Shi, Y et al. (2008) Cell Stem Cell 3(5):568-574)); and inhibitors of TGF-beta signaling (e.g., RepSox and the like (see, e.g. Ichida, J K. et al. (2009) Cell Stem Cell 5(5):491-503)), the disclosure related to reprogramming factors of which are herein incorporated by reference in their entirety. Examples of agents known in the art to promote cell reprogramming also include agents that reduce the amount of methylated DNA in a cell, for example by suppressing DNA methylation activity in the cell or promoting DNA demethylation activity in a cell. Examples of agents that suppress DNA methylation activity include, e.g., agents that inhibit DNA methyltransferases (DNMTs), e.g. 5-aza-cytidine, 5-aza-2′-deoxycytidine, MG98, S-adenosyl-homocysteine (SAH) or an analogue thereof (e.g., periodate-oxidized adenosine or 3-deazaadenosine), DNA-based inhibitors such as those described in Bigey, P. et al (1999) J. Biol. Chem. 274:459-44606, antisense nucleotides such as those described in Ramchandani, S et al, (1997) Proc. Natl. Acad. Sci. USA 94: 684-689 and in Fournel, M et al, (1999) J. Biol. Chem. 274:24250-24256, or any other DNMT inhibitor known in the art, the disclosure related to reprogramming factors of which are herein incorporated by reference in their entirety. Examples of agents that promote DNA demethylation activity include, e.g., cytidine deaminases, e.g., AID/APOBEC agents (N. Bhutani et al. (2009) Nature, December 21 [Epub ahead of print]; and K. Rai et al. (2008) Cell, 135:1201-1212), agents that promote G:T mismatch-specific repair activity, e.g. Methyl binding domain proteins (e.g. Mbp4) and thymine-DNA glycosylase (TDG) protein (K. Rai et al. (supra)), agents that promote growth arrest and DNA-damage-inducible 45 (GADD45) activity protein (K. Rai et al. (supra)), and the like, the disclosure related to reprogramming factors of which are herein incorporated by reference in their entirety.

In certain embodiments, the immortalizing factor may be human telomerase (hTERT), SV40 Large T antigen, HPV16 E6, HPV16 E7, or Bmil, or combinations thereof. Cellular immortalization by inhibiting the expression and/or function of the tumor suppressor p53 is well known in the art. However, compromising the natural mutation-suppressing function of p53 increases the presence of mutations in a cell and successive proliferation of the cell results in increased genetic variability between cells in the p53-immortalized population. Immortalization by methods using hTERT maintains the genetic stability of the immortalized cellular population by preserving the mutation-suppressing function of p53. In preferred embodiments, the immortalizing factor is hTERT.

In certain embodiments, conditional expression of the reprogramming, pluripotency and/or immortalizing factors disclosed herein can be under the control of inducible promoters such as a drug-inducible system (e.g., tetracycline-controlled transcriptional activation system, or an estrogen receptor-controlled transcriptional activation system) as well as through the use of tissue-specific promoters, all of which are well known in the art. In preferred embodiments, reprogramming and pluripotency factors are under the control of a tetracycline-controlled transcriptional activation system, expression of which is induced by exposing the cells to a tetracycline analog such as doxycycline.

In certain embodiments, expression of the reprogramming, pluripotency and/or immortalizing factors disclosed herein can be under the control of promoters that control expression in mammalian cells, including a promoter of the IE (immediate early) gene of cytomegalovirus (human CMV) or the initial promoter of SV40. An enhancer of the IE gene of human CMV may be used along with a promoter. A useful promoter can be the CAG promoter (comprising cytomegalovirus enhancer, chicken β-actin promoter and β-globin gene polyA signal site). In preferred embodiments, the immortalizing factor is under the control of a human CMV promoter. In certain embodiments, the cell(s) being reprogrammed are maintained on a feeder layer of irradiated mouse embryonic fibroblasts (MEFs). In certain embodiments, such cells are cultured in hES medium containing growth factors, such as βFGF. As described herein, hES medium may comprise basal media (DMEM-F12 Glutamax, 1×MEM-NEAA, 1×β-ME and 0.2×P/S) supplemented with 20% KSR and 8 ng/ml βFGF. In certain embodiments, the cell(s) being differentiated into fibroblasts were maintained in media comprising DMEM-F12 Glutamax, 1×NEAA, 1×β-ME, 0.2% P/S, 10% ES-FBS, and 8 ng/ml βFGF. Methods for the culture of somatic cells, iPS cells and cells being reprogrammed to iPSCs are well known in the art and media, feeder cells or matrices, and media supplements are commercially available. Moreover, media and culture conditions for differentiating iPSCs to various states, such as along endodermal, mesodermal, and ectodermal lineages are known in the art, and media and other factors are readily and/or commercially available.

In certain embodiments, the cells being reprogrammed are induced to express the reprogramming or pluripotency factors for at least 5, 10, 15, or 20 days. In preferred embodiments, the cells are induced to express the reprogramming or pluripotency factors for at least 21 days. In certain embodiments, secondary iPSCs and iMPCs derived from the reprogrammed immortalized secondary somatic cell can be maintained and expanded as described above, either on irradiated MEF in hES medium or in feeder-free conditions. In certain embodiments, cells are maintained and/or expanded and/or cultured on an alternative matrix. In certain embodiments, expression of markers indicative of pluripotency, such as Oct4 or TRA-1-60 (RNA or protein) are measured to assess when, over the course of multiple days following induction of expression of pluripotency factor(s), the cells have been reprogrammed to a pluripotent cell type (e.g., an iPSC).

Other reagents of interest for optional inclusion in the reprogramming technique are agents known in the art to promote the survival and differentiation of cells and include, for example, growth factors, supplements (e.g., B27 (Invitrogen)), nutrients (e.g., glucose), other protein such as transferrin, serum (e.g., fetal bovine serum, and the like), and the like.

### Methods of Use

In certain embodiments, the invention provides a method of assessing the effects of a test agent on nuclear reprogramming of a cell of the invention, in particular an immortalized secondary somatic cell or tertiary somatic cell, by contacting the cell with the test agent and assaying for a change in nuclear reprogramming in the cell relative to an untreated control cell not contacted with the test agent. In certain embodiments, the agent may cause the resulting cell to be less differentiated compared to the starting cell, for example, by reprogramming a somatic cell into an embryonic-like stem cell or iPSC. In certain embodiments, the agent may cause the resulting cell to have a different differentiated cell fate compared to the starting cell, for example, by directly reprogramming a somatic cell of a first type into a somatic cell of a second type without going through a de-differentiated pluripotent stage.

In certain embodiments, the invention provides a method of assessing the effects of a test agent on a cell of the invention (e.g., an immortalized secondary somatic cell, secondary iPSC, or tertiary somatic cell, as described herein) by contacting the cell with the test agent and assaying for a pharmacological or toxicological effect in the cell relative to a cell not contacted with the test agent. In certain embodiments, the effect is selected from cellular differentiation, proliferation, viability, and metabolism, or a combination thereof.

In certain embodiments, the invention provides a method of treating a subject in need of cellular therapy, comprising implanting a tertiary somatic cell as described herein into the subject, whereby the implanted cell restores, augments, or provides a desired function in the subject. In certain preferred embodiments, the method further comprises exposing a secondary iPSC or secondary iMPC to a differentiation agent(s) to produce the tertiary somatic cell for implantation.

In certain above embodiments utilizing tertiary somatic cells, the tertiary somatic cells comprise an inducible polycistronic exogenous nucleic acid encoding one or more reprogramming factors operably linked to a first regulatory sequence and a second exogenous nucleic acid encoding an immortalizing factor (e.g., as results from inducing expression of the reprogramming factors in an immortalized secondary somatic cell as described herein). In other above embodiments utilizing tertiary somatic cells, the tertiary somatic cells comprise an inducible polycistronic exogenous nucleic acid encoding one or more pluripotency factors operably linked to a first regulatory sequence and a second exogenous nucleic acid encoding an immortalizing factor (e.g., as results from differentiating a secondary iPSC or secondary iMPC as described herein).

The cells of the invention can be used to screen for agents (such as small molecule drugs, peptides, antibodies, and nucleic acids) or environmental conditions (such as culture conditions or other cues) that affect the characteristics of cells. Two or more drugs can be tested in combination (by exposing to the cells either simultaneously or sequentially), e.g., to detect possible drug-drug interactions and/or rescue effects (e.g., by testing a toxin and a potential anti-toxin). Drug(s) and environmental condition(s) can be tested in combination (by treating the cells with a drug either simultaneously or sequentially relative to an environmental condition), e.g., to detect possible drug-environment interaction effects.

In certain embodiments, the assay to assess treatment effects is selected in a manner appropriate to the cell type and agent and/or environmental factor being studied that include imaging, gene expression, and biochemical read-outs as disclosed in WO2002/04113, which is hereby incorporated by reference in its entirety. For example, changes in cell morphology may be assayed by standard light or electron microscopy, and/or through computer-assisted imaging techniques. Alternatively, the effects of agents or conditions potentially affecting the expression of cell surface proteins may be assayed by exposing the cells to either fluorescently labeled ligands of the proteins or antibodies to the proteins and then measuring the fluorescent emissions associated with each cell on the plate. As another example, the effects of agents or conditions which potentially alter the pH or levels of various ions within cells may be assayed using various dyes which change color at determined pH values or in the presence of particular ions. The use of such dyes is well known in the art. For cells which have been transformed or transfected with a genetic marker, such as the beta-galactosidase, alkaline phosphatase, or luciferase genes, the effects of agents or conditions may be assessed by assays for expression of that marker. In particular, the marker may be chosen so as to cause spectrophotometrically assayable changes associated with its expression.

In certain embodiments, the assay measures cellular proliferation, for example, by quantifying nuclei. Nuclear stains can also be used to enhance visualization and imaging of morphological characteristics. In certain embodiments, the number of nuclei in the process of mitosis (i.e., undergoing metaphase and anaphase) can also be identified and quantified. In certain embodiments, DNA synthesis can be measured as [3H]-thymidine or BrdU incorporation.

In some embodiments, the cells of the invention are used to screen pharmaceutical compounds for potential cytotoxicity (Castell et al., In: In Vitro Methods in Pharmaceutical Research, Academic Press, 375-410, 1997; and Cell Encapsulation Technology and Therapeutics, Kuhtreiber et al. eds., Birkhauser, Boston, Mass., 1999), which are herein incorporated by reference in their entirety. Cytotoxicity can be determined in the first instance by detecting and/or measuring the effect on cell viability, morphology, leakage of enzymes into the culture medium, and/or induction of apoptosis (indicated by cell rounding, condensation of chromatin, and nuclear fragmentation). In certain embodiments, cytotoxicity may be assessed by observation of vital staining techniques, ELISA assays, immunohistochemistry, and the like or by analyzing the cellular content of the culture, e.g., by total cell counts, and differential cell counts or by metabolic markers such as MTT and XTT.

Particular screening applications of the cells described herein relate to the testing of pharmaceutical compounds in drug research. The reader is referred generally to the standard textbook In Vitro Methods in Pharmaceutical Research, Academic Press, 1997, and U.S. Pat. No. 5,030,015. Cells of the invention may serve as test cells in standard drug screening and toxicity assays, as have been previously performed on cell lines or primary cells in short-term culture. Assessment of the activity of candidate pharmaceutical compounds generally involves combining the cells with the candidate agent, detecting and/or measuring any change in the morphology, marker phenotype, or metabolic activity of the cells that is attributable to the candidate compound (compared with untreated cells or cells treated with an inert compound), and then correlating the effect of the candidate agent with the observed change. The screening may be conducted because the candidate compound is designed to have a pharmacological effect on that particular cell type, or because a candidate compound designed to have effects elsewhere may have unintended side effects on the type of cell represented by the cell of the invention. Alternatively, libraries can be screened without any predetermined expectations in hopes of identifying compounds with desired effects.

In certain embodiments, selective labeling of one population with lipophilic dyes (e.g., carboxyfluorescein diacetate), nuclear stains (e.g., DAPI and Hoecht), or tagged proteins (e.g., GFP-tagged protein) can be used to distinguish cells in a population of interest from un-labeled cells.

Additional uses of the cells of the invention include, but are not limited to screening cytotoxic compounds, carcinogens, mutagens, growth/regulatory factors, pharmaceutical compounds, etc., in vitro; elucidating the mechanism of diseases and infections; studying the metabolism of a drug by detecting, identifying, and/or quantifying metabolites of the test agent; studying the mechanism by which drugs and/or growth factors operate; diagnosing and monitoring disease in a patient; and gene therapy, to name but a few.

### DEFINITIONS

The term “cell” is used herein in its broadest sense in the art and refers to a living body, which is a structural unit of tissue of a multicellular organism, surrounded by a membrane structure which separates the contents of the cell from the surrounding environment, and has genetic information and a mechanism for expressing it.

Cells used herein may be naturally occurring cells or artificially modified cells (e.g., fusion cells, genetically modified cells, etc.).

The term “somatic cell” as used herein may refer to all cells other than germ cells from mammals (e.g., humans, mice, monkeys, pigs, and rats). Examples of such somatic cells include keratinizing epithelial cells (e.g., keratinizing epidermal cells), mucosal epithelial cells (e.g., epithelial cells of the surface layer of tongue), exocrine epithelial cells (e.g., mammary glandular cells), hormone-secreting cells (e.g., adrenal medullary cells), cells for metabolism and storage (e.g., hepatocytes), boundary-forming luminal epithelial cells (e.g., type I alveolar cells), luminal epithelial cells of internal tubules (e.g., vascular endothelial cells), ciliated cells having a carrying capacity (e.g., airway epithelial cells), cells for secretion to extracellular matrix (e.g., fibroblasts), contractile cells (e.g., smooth muscle cells), cells of blood and immune system (e.g., T lymphocytes), cells involved in sensation (e.g., rod cells), autonomic nervous system neurons (e.g., cholinergic neurons), sense organ and peripheral neuron supporting cells (e.g., satellite cells), nerve cells and glial cells of the central nervous system (e.g., astroglial cells), chromocytes (e.g., retinal pigment epithelial cells), and progenitor cells thereof (tissue progenitor cells). Without particular limitation concerning the degree of cell differentiation, the age of an animal from which cells are collected, or the like, both undifferentiated progenitor cells (also including somatic stem cells) and terminally-differentiated mature cells can be similarly used as origins for somatic cells in the present invention. Examples of undifferentiated progenitor cells include tissue stem cells (somatic stem cells) such as neural stem cells, hematopoietic stem cells, mesenchymal stem cells, and dental pulp stem cells.

“A cell of the invention” or “cells of the invention” as used herein mean a cell, cells, and/or a population of cells as described herein and/or obtainable by methods described herein.

As used herein, “subject” means a human or animal (in the case of an animal, preferably a mammal). In one aspect, the subject is a human.

As used herein, “cellular differentiation” or “differentiation” is the process by which a less specialized cell becomes a more specialized cell type.

As used herein, the term “stem cell” refers to a cell capable of giving rising to at least one type of a more specialized cell. A stem cell has the ability to self-renew, i.e., to go through numerous cycles of cell division while maintaining the undifferentiated state, and has potency, i.e., the capacity to differentiate into specialized cell types. Typically, stem cells can regenerate an injured tissue. Stem cells herein may be, but are not limited to, embryonic stem (ES) cells, induced pluripotent stem cells, or tissue stem cells (also called tissue-specific stem cell, or somatic stem cell). Any artificially produced cell which can have the above-described abilities (e.g., fusion cells, reprogrammed cells, or the like used herein) may be a stem cell.

“Embryonic stem (ES) cells” are pluripotent stem cells derived from early embryos, as is well understood in the art.

Unlike ES cells, tissue stem cells have a limited differentiation potential. Tissue stem cells are present at particular locations in tissues and have an undifferentiated intracellular structure. Therefore, the pluripotency of tissue stem cells is typically low. Tissue stem cells have a higher nucleus/cytoplasm ratio and have few intracellular organelles. Most tissue stem cells have low pluripotency, a long cell cycle, and proliferative ability beyond the life of the individual. Tissue stem cells are separated into categories, based on the sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by inserting certain genes, referred to as reprogramming factors.

“Pluripotent” as used herein refers to a cell that has the capacity to differentiate to cells of all three germ layers (endoderm, ectoderm, and mesoderm). Pluripotent cells are also self-renewing. Embryonic stem cells and induced pluripotent stem cells are art recognized examples of pluripotent cells. In certain embodiments, any of the iPSC cells, including iPSC cells differentiated from a secondary or tertiary somatic cell of the disclosure may be defined using any one or combination of the characteristics provided herein. A pluripotent cell is characterized by expression of certain markers (genes and/or antigens) recognized in the art, and these may vary between different species (e.g., mouse versus humans). In certain embodiments, a pluripotent cell expresses one or more markers selected from Oct 4, TRA-1-60, DNMT3B, LIN28A and REX1 (e.g., the cell comprises expression of one or more such markers). In certain embodiments, a pluripotent cell expresses all of these markers. In certain embodiments, a pluripotent cell expresses Oct4. In certain embodiments, a pluripotent cell expresses TRA-1-60 antigens. In certain embodiments, a pluripotent cell expresses Oct4 and one or more (1, 2, 3, or 4) markers selected from: alkaline phosphatase, SSEA-4, GDF3, and NANOG (e.g., the cells comprises expression of one or more such markers). Optionally, the pluripotent cell further expresses one or more (1, 2, or 3) of: TRA-1-60, DNMT3B, LIN28A and REX1. In certain embodiments, marker expression comprises gene expression, such as expression as measured by RT-PCR or RNA-Seq. In certain embodiments, marker expression comprises protein expression, as measured by immunocytochemistry using an antibody that specifically binds the protein. In certain embodiments, marker expression is measured in pluripotent stem cells (such as embryonic and induced PSC) cultured using standard conditions as described in C. A. Gifford et al. (2013) Cell, 153: 1149-1163. In certain embodiments, marker expression is measured using a method described herein.

“Multipotent cell” as used herein refers to a cell that has the capacity to differentiate into more than one cell type (e.g., a subset of the cell types of an organism). Unlike a pluripotent cell, a multipotent cell does not have the capacity to differentiate into cells of all three germ layers, but may be capable of giving rise to multiple different types of, for example, ectodermal or mesodermal cell types.

“Differential expression” refers to an increased, up-regulated or present, or decreased, down-regulated or absent, gene expression as detected by the absence, presence, or a Bayesian statistic (greater than 0), which corresponds to a significant difference in the amount of transcribed messenger RNA or translated protein in a sample.

“High-throughput screening” (HTS) refers to a process that uses a combination of modern robotics, data processing and control software, liquid handling devices, and/or sensitive detectors, to efficiently process a large amount of (e.g., thousands, hundreds of thousands, or millions of) samples in biochemical, genetic or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process one can rapidly identify active compounds, small molecules, antibodies, proteins or polynucleotides which modulate a particular biomolecular/genetic pathway. The results of these experiments provide starting points for further drug design and for understanding the interaction or role of a particular biochemical process in biology.

The term “treating” is art-recognized and includes administration to the host of one or more of the subject compositions, e.g., to diminish, ameliorate, or stabilize the existing unwanted condition or side effects thereof.

### EXAMPLES

### Example 1

### Generation of a Human Secondary Reprogramming System for High-Throughput Analyses

FIG. 2 provides a schematic representation of the methods used to produce the immortalized secondary somatic cells and secondary iPSCs described in this example.

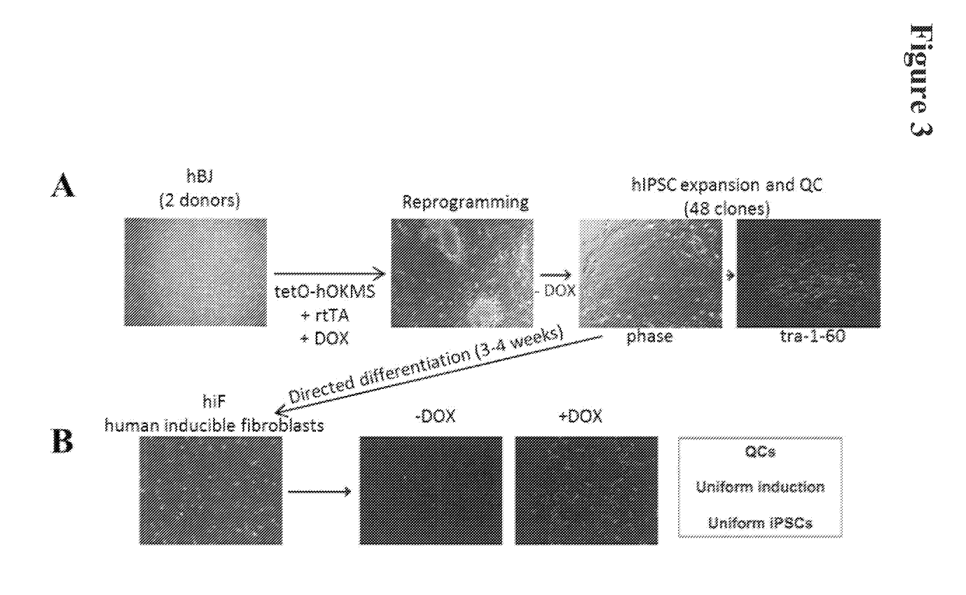

For primary reprogramming, human BJ neonatal foreskin fibroblasts (Stemgent) were infected with two lentiviruses carrying the Tet-On Advanced transactivator (FUW-M2rtTA; Addgene Plasmid 20342) and a doxycycline (DOX) inducible OCT4-KLF4-MYC-SOX2 ORF. This second construct was obtained by cloning the polycistronic ORF (Addgene Plasmid 27512) in a DOX inducible vector (FUW-tetO; Addgene Plasmid 20725). After recovery, BJ-infected fibroblasts were then plated on irradiated MEF feeders (Globalstem) and cultured in presence of doxycycline (1 ug/ml) for 21 days in a reprogramming media (20% KSR, 1×NEAA, 1×β-ME, 0.2% P/S, in DMEM/F12 Glutamax, 8 ng/ml bFGF—Lifetech) (FIG. 3A).

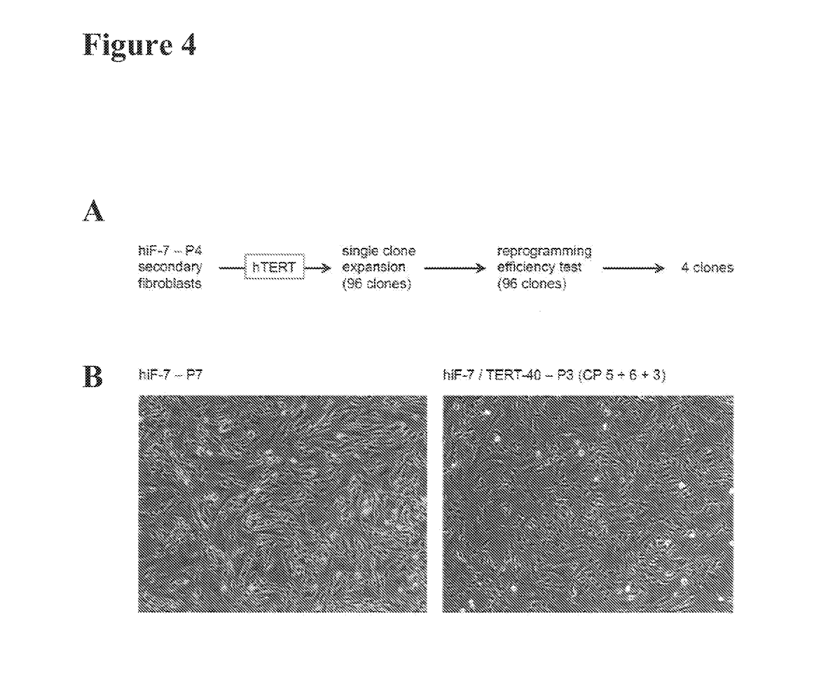

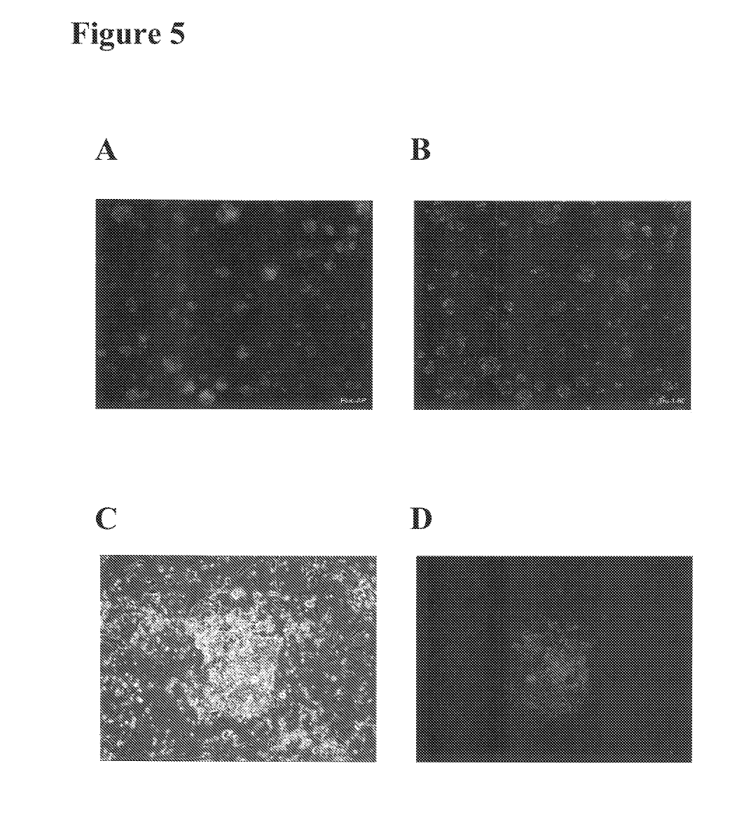

Individual clones were picked and expanded in the reprogramming media. 48 clones were expanded and tested for their differentiating potential into fibroblasts by culturing for 21 days in fibroblasts media (hiF media—10% ES-FBS, 1×NEAA, 1×β-ME, 0.2% P/S, in DMEM/F12 Glutamax, 8 ng/ml bFGF—Lifetech). The same cells were then tested by immunofluorescence for absence of the reprogramming factors in absence of doxycycline, and their expression upon supplementation of 1 ug/ml of doxycycline (FIG. 3B). This restricted the selection to three clones (human-induced-fibroblasts clones, hif-clones). After five more passages from the initial differentiation, pre-senescent cells were observed by b-gal senescent staining (SIGMA). The hif clone number 7 (hif-7) were subsequently immortalized by infecting with a CMV-hTERT lentivirus (Abmgood Bioscience). Cells were selected using 1 ug/ml of puromycin and plated to allow a clonal selection of immortalized hif-7 clones (FIG. 4 top). 96 clones were picked and tested for both extended lifespan and reprogramming capacity upon doxycycline supplementation in reprogramming media on irradiated MEF feeders. Three clones were selected (hif-TERT) and the clone hif-TERT-40 was extensively characterized by karyotype (cell line genetics), absence of senescence (SIGMA) and consistent reprogramming efficiency for 40 passages after clonal selection (4 months of continuous culturing). Images of clone hif-TERT-40 cells are shown at passage 2 and passage 15 in the bottom right and left panels, respectively, in FIG. 4. Reprogramming efficiency was detected by both immunofluorescence for tra1-60 antigen (Stemgent) (FIGS. 5B and 5D) and alkaline phosphatase staining

(VectorLaboratories) (FIG. 5A). A corresponding phase contrast image of FIG. 5D is shown in FIG. 5C.

### Example 2

### Robust Reprogramming Efficiency of Immortalized Secondary Somatic Cells

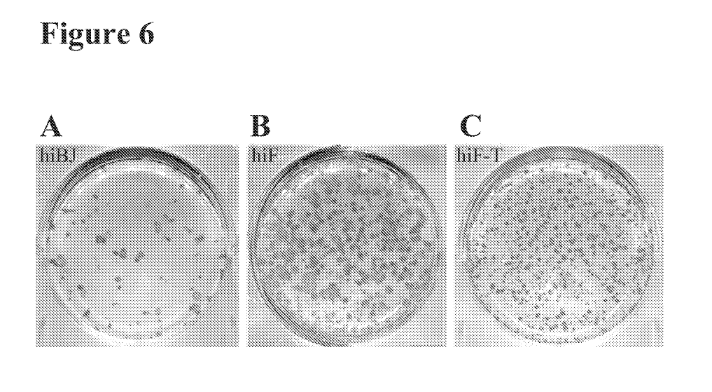

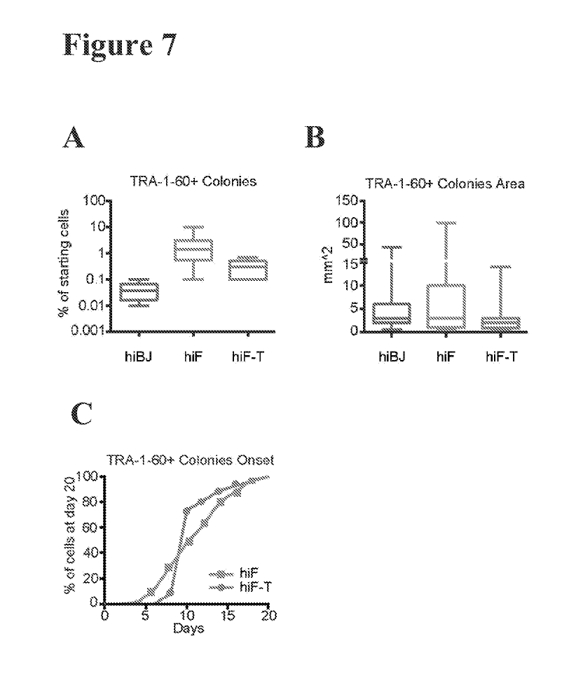

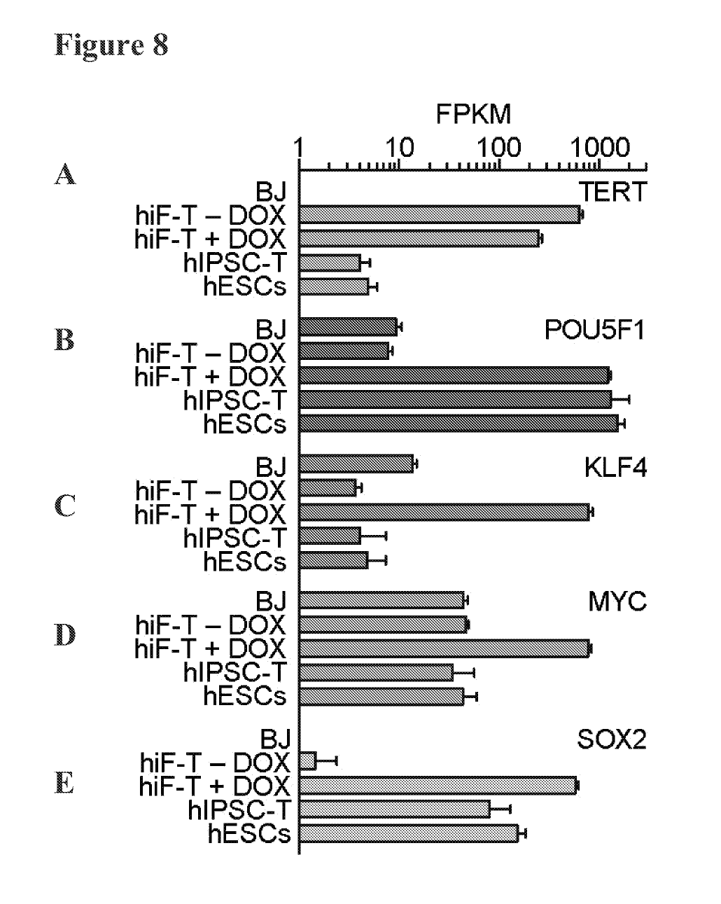

Following induction of pluripotency factors OCT4, KLF4, MYC, and SOX2 via an inducible polycistronic nucleic acid, both primary human BJ (hiBJ) cells and secondary somatic (hiF) cells generated colonies of reprogrammed cells that were highly heterogeneous in size and appeared asynchronously over the course of three weeks following induction of the polycistronic pluripotency factors (FIGS. 6A and 6B and FIG. 7). Surprisingly, colonies of reprogrammed cells generated from immortalized secondary somatic (hiF-T) cells were far more homogeneous in terms of both their size and their onset of appearance over the course of three weeks following induction of the polycistronic pluripotency factors OCT4, KLF4, MYC, and SOX2 (FIG. 6C and FIG. 7). Moreover, reprogramming of immortalized secondary somatic (hiF-T) cells gave rise to secondary iPSCs (hIPSC-T) that expressed similar levels of Oct4 (POU5F1), KLF4, Myc and SOX2 as reference hESCs (FIG. 8). Secondary iPSCs (hIPSC-T) also exhibited decreased expression of hTERT, relative to its expression in immortalized secondary somatic (hiF-T) cells, suggesting that hTERT expression was down-regulated during the reprogramming process. This is consistent with the expected downregulation of the CMV promoter, which drives TERT expression in this experiment, in pluripotent stem cells.

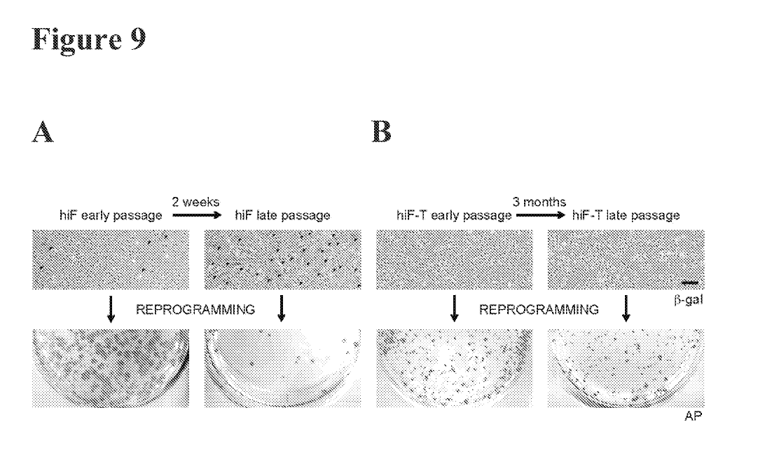

In addition, secondary somatic cells (hiF cells) rapidly lost their reprogramming potential with successive passages in culture (FIG. 9A; bottom panel), which correlated with the appearance of senescent cells (FIG. 9A; top panel). In contrast, immortalized secondary somatic cells (hiF-T cells) did not display senescence (FIG. 9B; top panel) and consequently showed efficient, reproducible reprogramming kinetics even after three months in continuous culture (FIG. 9B; bottom panel).

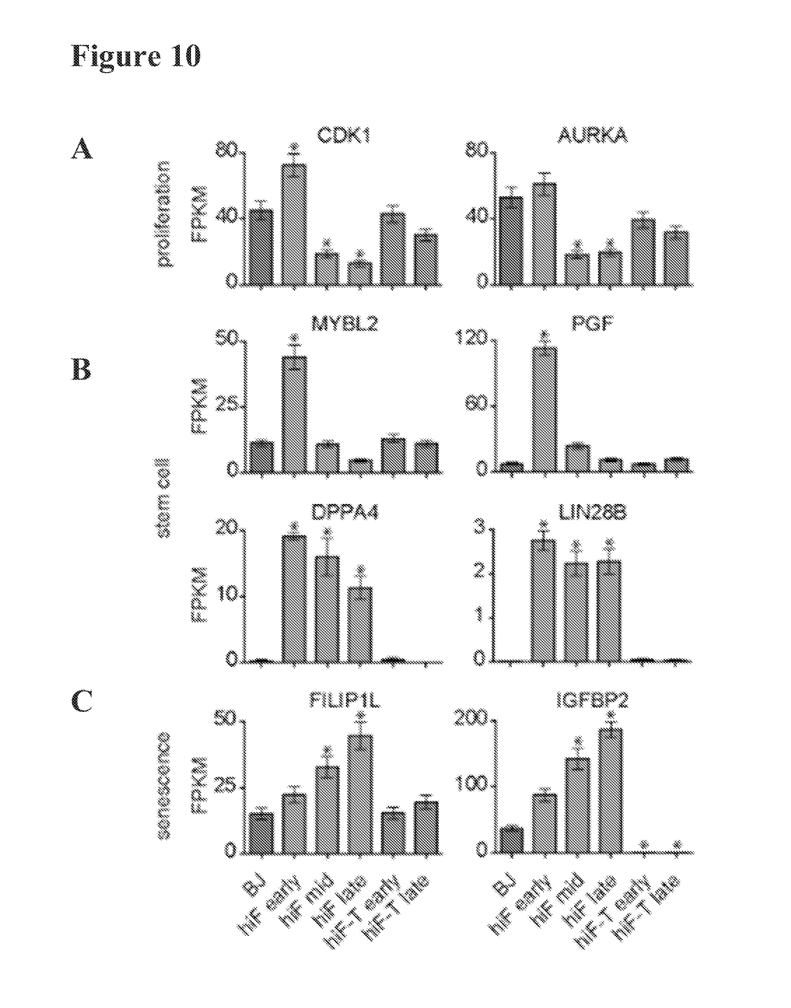

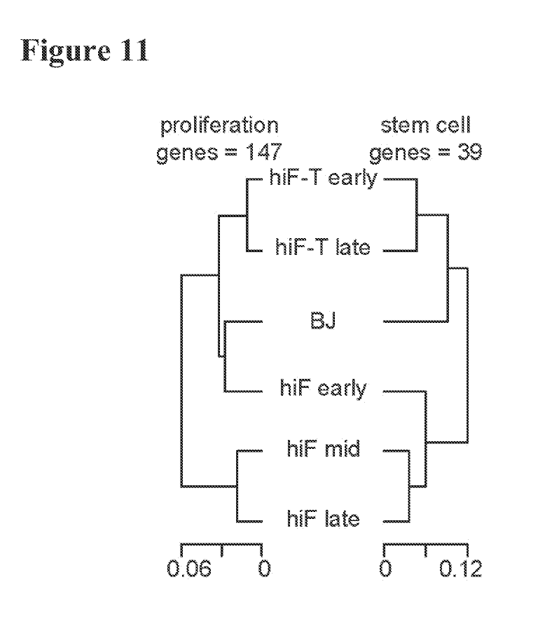

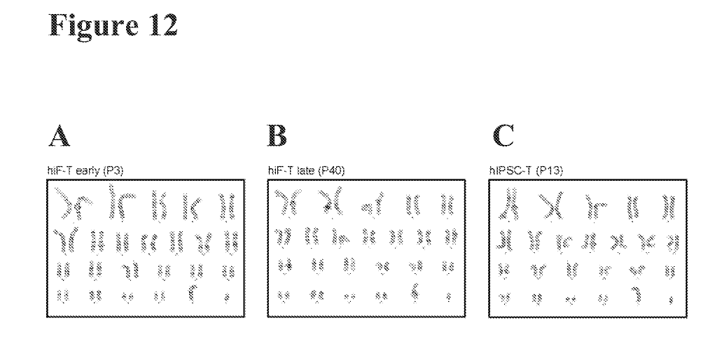

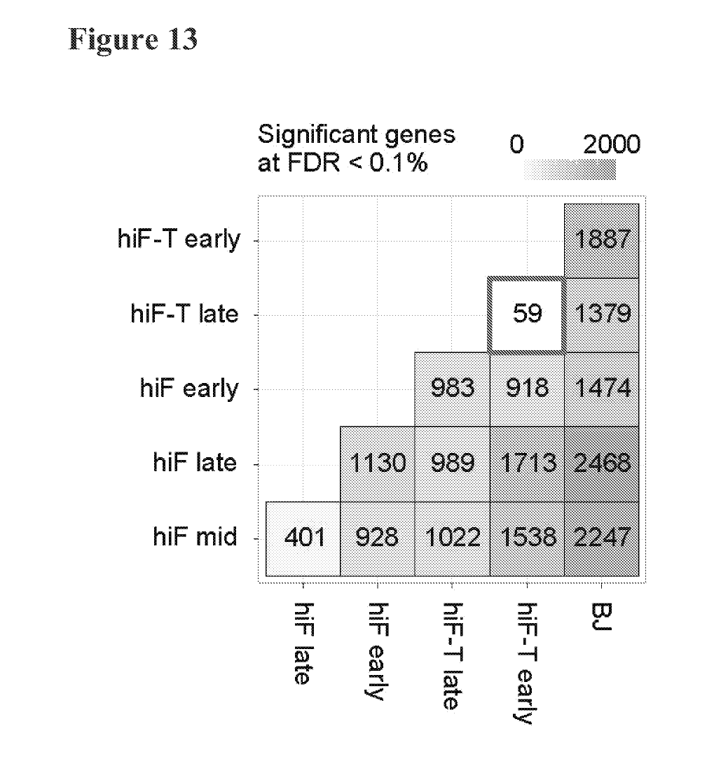

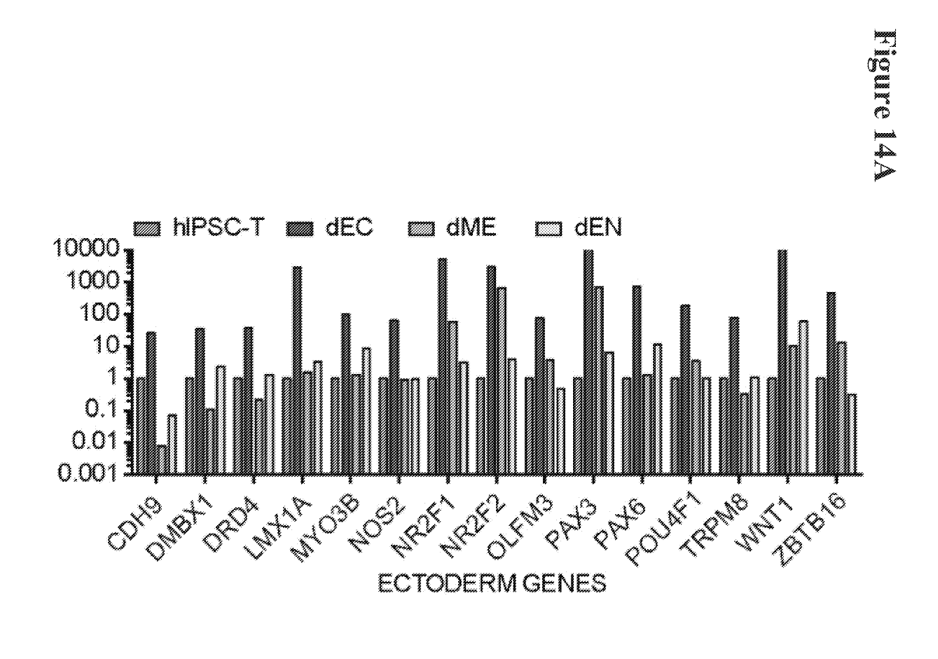

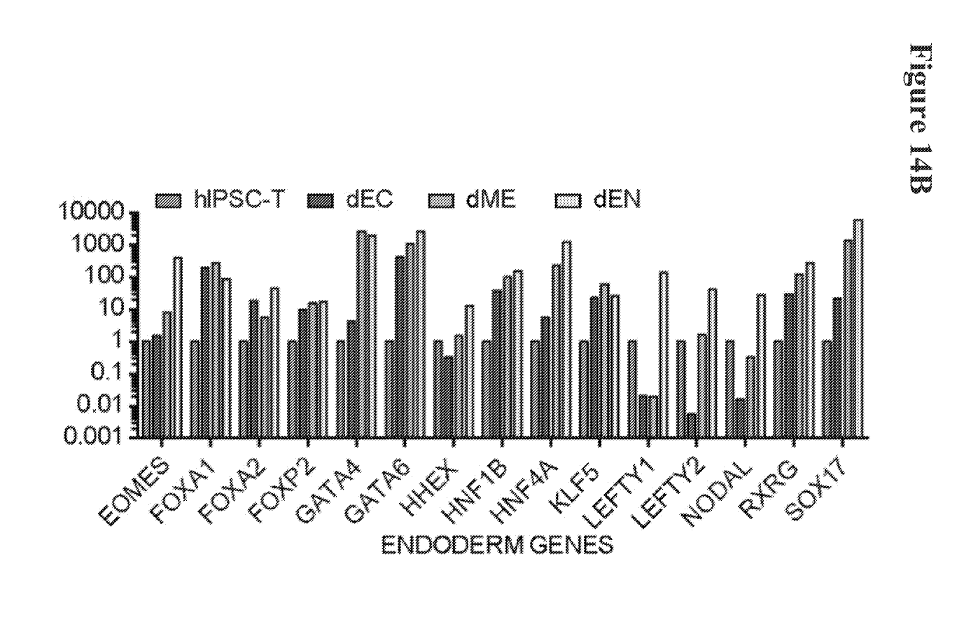

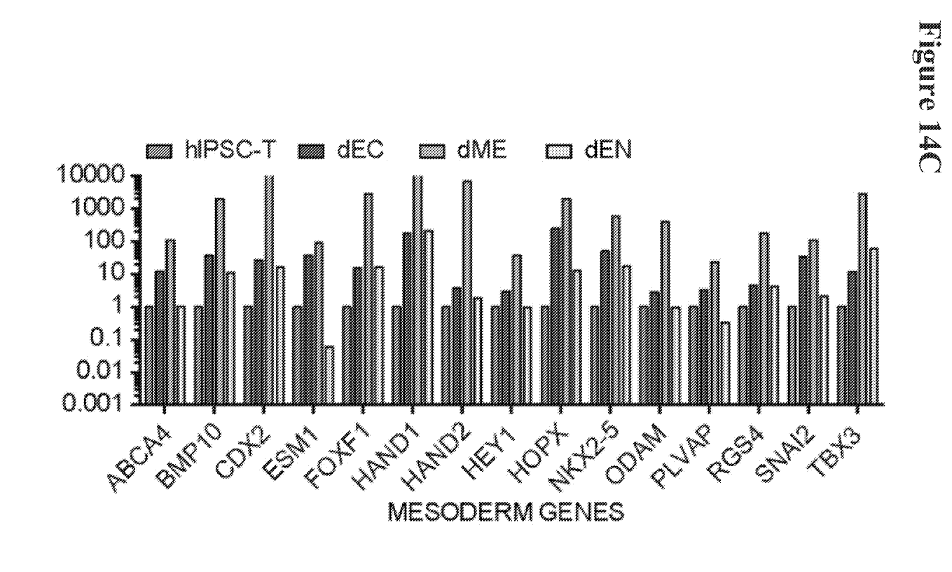

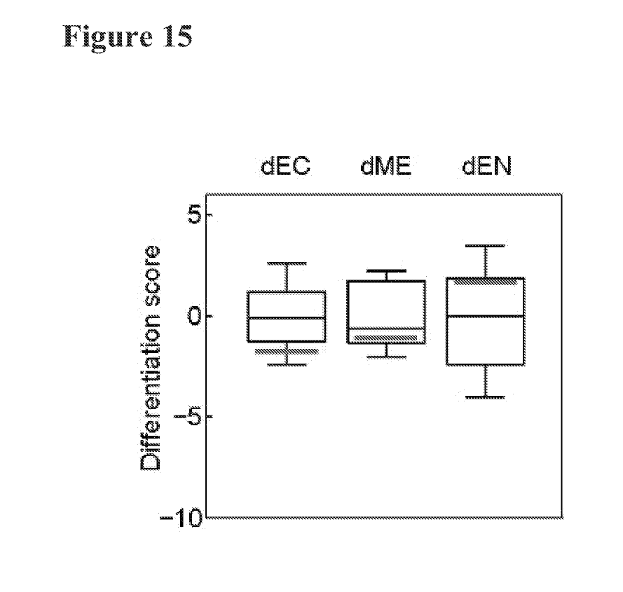

Expression profiling by RNA sequencing (RNA-seq) of the fibroblast-like cells showed that secondary somatic (hiF) cells rapidly down-regulated proliferative genes, e.g., CDK1 and AURKA, after limited passaging, while immortalized secondary somatic (hiF-T) cells maintained consistent expression of these genes over long-term culture (FIG. 10A). Furthermore, secondary somatic (hiF) cells expressed high levels of common stem cell genes (e.g., MYBL2, PGF, DPPA4 and LIN28B), even as they approached senescence indicated by the expression of senescence genes, e.g., FILIP1L and IGFBP2. In contrast, the immortalized secondary somatic (hiF-T) cells were maintained in culture long enough to fully silence the same stem cell-related genes (FIGS. 10B and 10C). Furthermore, prevention of senescence by hTERT expression in immortalized secondary somatic (hiF-T) cells enabled these cells to be maintained in culture long enough to acquire a fully somatic expression profile (FIG. 11). Without being bound by theory, this uniform and fully somatic nature observed in these immortalized secondary somatic (hiF-T) cells may underlie their demonstrated homogeneity of reprogramming. Importantly, the immortalized secondary somatic (hiF-T) cells also showed high levels of genomic stability based on karyotype analysis (e.g., maintaining a normal karyotype; FIG. 12) and transcriptional stability over prolonged periods of culture (FIG. 13). Moreover, secondary iPSCs (hIPSCs) derived from immortalized secondary somatic (hiF-T) cells (also known as hIPSC-T) maintained their capacity to give rise to cells of all three germ layers (ectoderm, endoderm, and mesoderm) in vitro (FIG. 14), at levels that were highly similar to reference embryonic and induced pluripotent stem cells (PSCs) (FIG. 15).

### Example 3

### Comprehensive Analyses of Stages in Reprogramming Using Immortalized Secondary Somatic Cells

The increased proliferative capacity of immortalized secondary somatic (hiF-T) cells was leveraged to generate large numbers of cells for comprehensive immunophenotypic and transcriptomic analyses at all stages of the reprogramming process. A benefit of the invention is the ability to generate large numbers of these cells which are robust and can be used in research and drug screening assays. Upon induction of a reprogramming factor(s), in this example the pluripotency factors Oct4, Klf4, Myc, and Sox2 (OKMS), immortalized secondary somatic (hiF-T) cells rapidly and homogeneously lost the somatic cell marker CD13, which was followed by the expression of the embryonic marker SSEA-3, and later, the expression of the pluripotency-associated marker TRA-1-60 antigen.

RNA-Seq analysis showed a continuous trajectory of transcriptional changes beginning at the immortalized secondary somatic (hiF-T) state and ending with fully established secondary iPSC (hIPSC-T) cells. Clustering analysis, comparison with reference hESC signatures and gene ontology analysis revealed that, similar to murine systems (T. S. Mikkelsen et al. (2008) Nature, 454: 49-55; J. M. Polo et al. (2012) Cell, 151: 1617-1632; and E. M. Chan et al. (2009) Nat. Biotechnol., 27: 1033-1037), OKMS induction led to the immediate down-regulation of mesenchymal signature genes (e.g. SNAI2). Pluripotency-related genes were subsequently activated in two waves, with core regulators like LIN28A (E. M. Chan et al. (supra)) fully activated by day 20. A final set of genes which peaks after derivation of a hIPSC-T from reprogrammed (TRA-1-60+) colonies likely reflects a commitment towards neuro-ectoderm and epiblast, which is characteristic of standard pluripotent stem cell culture conditions (P. J. Tesar et al. (2007) Nature, 448: 196-199).

While rapid down-regulation of somatic genes and subsequent activation of the pluripotency network have been described (I. H. Park et al. (2008) Nature, 451: 141-146; and E. M. Chan et al. (supra)), characterization of the transition between these states has been limited by the dramatic heterogeneity of previous human systems. Here, we found that the strikingly homogeneous kinetics of hiF-T cells (e.g., within a given culture or generated from a given clone) permitted robust isolation and characterization of these cells and exemplified the benefits of the methods and cells of the disclosure. Analysis of the hiF-T cells revealed several transient waves of gene activation that were unique to the reprogramming process and absent in secondary iPSC cells. These transient waves allowed identification of the trajectories responsible for acquisition of pluripotency.

Analysis of miRNA expression reinforced this model. We similarly observed the rapid loss of expression of somatic miRNAs, followed by up-regulation of miRNAs under developmental control and eventually of pluripotency markers. Strikingly, while many miRNAs were highly expressed in hiF-T cells, more than 50% of miRNAs detected at the end of the reprogramming process were from a small number of non-somatic families.

### Example 4

### Use of Immortalized Secondary Somatic Cells in an RNAi Screen to Identify Reprogramming Regulators

An RNAi screen was performed using a pooled lentiviral library encoding ˜2,900 shRNAs targeting 370 distinct epigenetic regulators (a sub-pool of the human 45K shRNA pool used in B. Luo et al. (2008) Proc. Natl. Acad. Sci. U.S.A., 105: 20380-20385. Expression of the shRNAs was under the control of the constitutive U6 snRNA promoter in the lentiviral pLKO.1 vector. shRNA pool production and infection conditions were performed as previously described in B. Luo et al., (supra).

An estimated starting number of 6×10⁷ immortalized secondary somatic (hiF-T) cells was determined to be required to quantitatively screen the activity of every member in the pool based upon the following specific considerations: (a) the number of hairpins in the pool (×2,900); (b) the <50% infection efficiency required to ensure ˜1 shRNA per cell (×2); (c) the requirement of ˜50 independent integrations of each hairpin in the reprogrammed cell population (×50); and (d) the 0.5% immortalized secondary somatic (hiF-T) cell basal reprogramming efficiency (×200).

Immortalized secondary somatic (hiF-T) cells were collected 1 week after infection with the shRNA encoded lentivirus in the absence of doxycycline and subsequently reprogrammed for 15 days in the presence of doxycycline to induce expression of pluripotency factors. The resulting TRA-1-60+ fraction was collected. The relative enrichment of each hairpin in the reprogrammed cells relative to the control cells was estimated by retrieving the shRNA pool by PCR and then sequencing the resulting amplicons as previously described in Strezoska et al., (2012) PLoS ONE, 7(8), e42341. doi:10.1371/journal.pone.0.0042341.

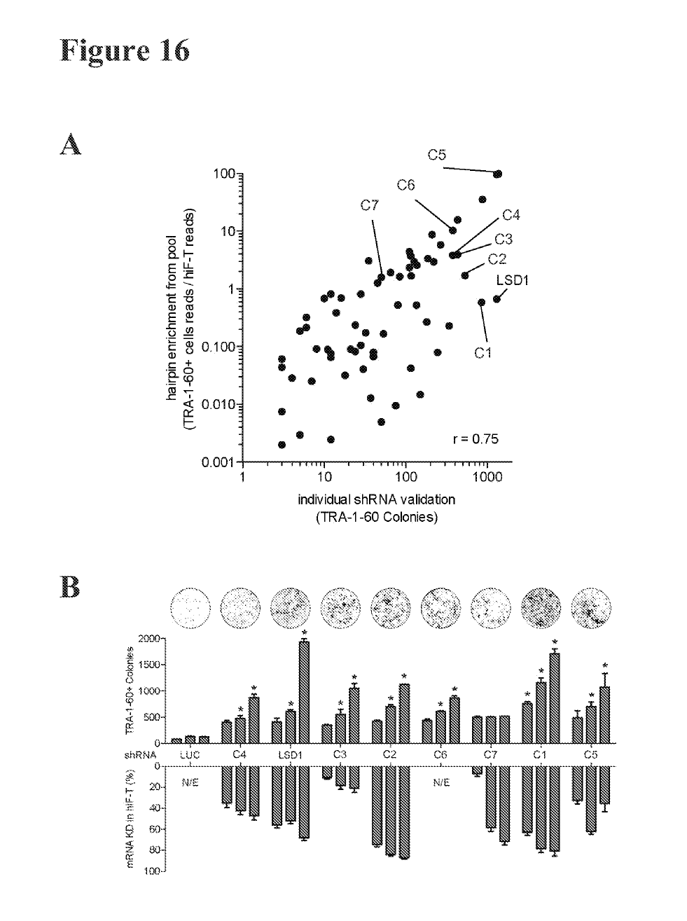

Twenty-three candidate genes for which shRNA-mediated knock-down improved reprogramming of immortalized secondary somatic (hiF-T) cells were identified by comparing shRNA abundance before (“hiF-T”) and after (“TRA-1-60+”) reprogramming using deep sequencing (FIG. 16A). The on-target effects of eight of the twenty-three candidate genes were validated, which included seven candidates C1-C7 and LSD1, a histone lysine demethylase, inhibition of which was previously reported to enhance reprogramming (FIG. 16B) (T. T. Onder et al. (2012) Nature, 483: 598-602; and W. Li et al. (2009) Stem Cells, 27: 2992-3000). These results demonstrate that immortalized secondary somatic cells provide a robust resource of large numbers of cells suitable for studying reprogramming and for screening.

Target sequences of the shRNAs targeting LSD1 (KDM1A) and the control luciferase (LUC) were:

| Gene | TRC Public ID | Target Sequence |
|---|---|---|
| LUCIFERASE | TRCN0000072253 | ACACTCGGATATTTGATATGT |
| LUCIFERASE | TRCN0000072259 | CGCTGAGTACTTCGAAATGTC |
| LUCIFERASE | TRCN0000072256 | ACGCTGAGTACTTCGAAATGT |
| KDM1A | TRCN0000327932 | CCACGAGTCAAACCTTTATTT |
| KDM1A | TRCN0000046070 | CCAACAATTAGAAGCACCTTA |
| KDM1A | TRCN0000046068 | GCCTAGACATTAAACTGAATA |

### Example 5

### Reprogramming of Human Fibroblasts into Muscle Cells Using myoD in a hTERT Background

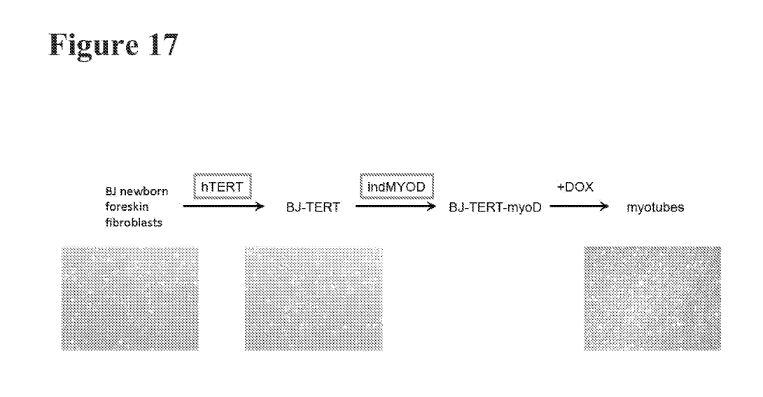

Human BJ neonatal foreskin fibroblasts (Stemgent) were infected with a CMV-hTERT lentivirus (Abmgood Bioscience) (FIG. 17, left panel). Cells were selected using 1 ug/ml of puromycin and plated to allow a clonal selection of immortalized clones (BJ-TERT; FIG. 17, middle panel). Cells were then infected with two lentiviruses carrying the Tet-On Advanced transactivator (Addgene Plasmid 20342) and a doxycycline (DOX) inducible MYOD ORF. Individual clones were picked and expanded for further manipulation. Cells were induced to form myotubes in the presence of doxycycline (1 ug/ml) in a muscle media (alphaMEM, 2% horse serum, 0.2% P/S—Lifetech). The resulting cells resembled muscle cells that were capable of forming myotubes (FIG. 17, right panel).

### Methodologies: Unless Otherwise Indicated, the Following Methods are Used Throughout the Examples and are Exemplary of Suitable Methods.

### A. Cell Culture and Reprogramming

The inducible, polycistronic human OKMS lentiviral vector (Addgene 70808) was generated by cloning an OKMS polycistron spaced by 2A peptides (Addgene 27512) into the FUW-tetO backbone (Addgene 20725). FUW-M2rtTA (Addgene 20342) was used in combination with the OKMS lentiviral vector to provide the reverse tetracycline transactivator.

All cell culture reagents were purchased from Life Technologies, unless otherwise specified. Primary BJ human foreskin fibroblasts (Stemgent) were expanded on gelatin coated dishes in hiF medium composed of basal medium (DMEM-F12 Glutamax, 1×MEM-NEAA, 1×beta-ME, 0.2×P/S) supplemented with 10% ES-FBS and 16 ng/ml FGFbasic. BJ cells were co-infected with OKMS and M2rtTA lentivectors at MOI ˜1.

All the reprogramming experiments were performed seeding fibroblasts on irradiated MEF (murine embryonic fibroblasts; a feeder layer; GlobalStem), inducing OKMS by DOX supplementation (2 ug/ml) for the indicated times, in hiF medium for the first 2 days and then in hES medium (basal medium supplemented with 20% KSR and 8 ng/ml βFGF). After ˜21 days of reprogramming, IPSC colonies were picked and expanded without DOX, either on irradiated MEF in hES medium or in feeder-free conditions with either geltrex matrix in mTeSR1 medium (Stemcell technologies) or geltrex/vitronectin in E8 medium. IPSC colonies generated from reprogramming of primary BJs were used to generate secondary hiF cells by directed differentiation in hiF medium (either embryoid bodies or on-plate colonies differentiation) (E. M. Chan et al., (supra); I. H. Park et al., (supra)). hiF cells were considered differentiated and used for reprogramming experiments (as passage 1, P1) no earlier than 5 weeks after the switch to hiF medium.

Immortalization of hiF cells was performed using a constitutive hTERT lentivirus co-expressing the Puromycin (PURO) resistance gene (abmgood). Derivative hiF-T cells were selected with PURO (1 μg/ml) and plated at high dilution rate to isolate and expand individual clones. Each clone was expanded for approximately 3 weeks before collecting enough cells for freezing and line establishment (as passage 1-P1).

hiF and hiF-T cells were passed every 3 days, using a splitting rate of 1:3. For comparative analyses, the following collection passages were used: BJ cells (P6-P9), hiF cells (early <P6, mid P6-P8, late >P8), hiF-T (early <P20, late >P30). hiF-T cells of at least passage 15 were used for standard reprogramming experiments, seeding 2-4×10⁴ cells/cm² on >75% confluent MEF layer. At any given time point of reprogramming, cells were dissociated in Accutase and MEF depleted by magnetic beads separation (Milteny Biotec). Eluates were then collected either as total fractions or further magnetic beads separation was applied to isolate SSEA3 or TRA-1-60 positive or negative sub-populations. Fraction purity of at least 95% was validated by flow cytometry.

Directed differentiation of hIPSC-T was performed as previously reported (C. A. Gifford et al. (supra)) and differentiation potential was quantified by the qRT-PCR Scorecard approach (Bock et al. (2011) Cell, 144: 439-452) with some modification (Bock, Tsankof and Messiner personal communication, Life Technologies). Reference hESC (H1, H9 and HUES clones 1, 3, 8, 9, 13, 28, 44, 45, 48, 49, 53, 62, 63, 64, 65, 66) and hIPSC (clones 11b, 11c, 15b, 17a, 17b, 18, 18b, 18c, 20b, 27b, 27e, 29e) were obtained from the Harvard Stem Cell Institute IPSC Core.

### B. Cytochemistry, Immunostaining and Flow Cytometry

Chromogenic staining was performed according to manufacturer specifications, using Red Alkaline phosphatase (Vector Labs), senescence-associated beta-galactosidase (Sigma) and TRA-1-60 (P. D. Manos et al. (2011) Curr Protoc Stem Cell Biol, Chapter 1, Unit 1C.12-1C.12.14). Cytogenetic analysis of metaphasic chromosomes was performed by standard G-band karyotyping (Cell Line Genetics).

Immunofluorescence and flow cytometry analyses were performed as previously described (C. A. Gifford et al. (supra)) using the following antibodies: TRA-1-60, SSEA3, CD13 (Biolegend), POU5F1 (cat. 611202, BD Biosciences), UTF1 (cat. 105090, Abcam), DPPA3 (cat. sc-376862, Santa Cruz Biotech.). Flow cytometry data were processed using FlowJo and immunofluorescence image processing and measurements were performed using ImageJ (C. A. Schneider et al. (2012) Nat. Methods, 9: 671-675).

### C. Transcriptomic Analyses

Total RNA, including the small RNA fraction, was obtained by organic extraction followed by miRNeasy purification (Qiagen). RNA-Seq Illumina libraries for mRNA profiling were prepared from 100 ng total RNA using the TruSeq RNA Sample Prep Kit v2 (Illumina) and small RNA libraries were prepared from 1 ug of total RNA using the TruSeq Small RNA Sample Prep Kit (Illumina).

mRNA-Seq libraries were sequenced with approximately 20 million 100 bp paired end reads each. Cell fractions were sequenced at least in biological duplicate. mRNA-Seq reads were analyzed with the Tuxedo Tools following a standard protocol (C. Trapnell et al. (2012) Nature Protocols, 7: 562-578), excluding transcriptome assembly (Alternative Protocol “B”). Reads were mapped with TopHat version 2.0.7 and Bowtie version 2.1.0 with default parameters against build hg19 of the human genome, and build 13 of the GENCODE human genome annotation (J. Harrow et al. (2012) Genome Res., 22: 1760-1774). Samples were quantified with the Cufflinks package version 2.2.0. Differential expression was performed using Cuffdiff 2.2.0 with default parameters.

Small RNA libraries were analyzed similarly to mRNA-Seq libraries. Small RNA read were mapped to the human genome with TopHat 2.0.7 and Bowtie 2.1.0. Spliced alignment by TopHat was disabled by omitting the GENCODE annotation file and providing the -no-novel-juncs option, which forces all alignments to be unspliced. Sequencing adapters were clipped from the reads using fastx_clipper form the FASTX toolkit (http://hannonlab.cshl.edu/fastx_toolkit/), using the options “-a TGGAATTCTCGGGTGCCAATGAACTCCAG-1 18-Q 33” Reads were mapped with TopHat, as opposed to Bowtie, because the resulting alignment files are properly formatted for direct input to Cuffdiff. Small RNA expression levels were estimated using Cuffdiff 2.2.0 by providing Cuffdiff with the small RNA records from the GENCODE annotation (biotypes miRNA, miRNA_pseudogene, misc_RNA, misc_RNA_pseudogene, snRNA, snRNA_pseudogene, snoRNA, snoRNA_pseudogene. Piwi-interacting RNAs (piRNAs) were included in the annotation by directly mapping Illumina-supplied piRNA sequences to the genome, then converting these alignments to GTF records using a custom script. The number of reads generated from each small RNA is directly proportional to abundance, and independent of small RNA length, so the option -no-length-correction was provided to Cuffdiff, disabling length-based normalization of read counts.

Further analysis of expression data, such as MDS clustering of samples and k-medoids clustering of genes based on expression profile was performed with CummeRbund 2.0.0. Differential expressed (DE) genes in gene matrixes were calculated at the indicated False Discovery Rate (FDR—after Benjamini-Hochberg correction) and used to compute Gene Set Enrichment Analyses. Gene Sets used for enrichment analyses were obtained from Molecular Signatures Database v4.0 (A. Subramanian et al. (2005) Proc. Natl. Acad. Sci. U.S.A., 102: 15545-15550) (FIG. 10) or REACTOME (D. Croft (2013) Methods Mol. Biol., 1021: 273-283) (FIG. 16). Given the high number of DE genes along the reprogramming trajectory, we set additional filters to obtain DE genes to build the k-medoids clustering. To be included in the gene list, each gene needed to show an FPKM expression of at least 5 in at least one time point, and a log 4 fold change in at least one pairwise comparison. The enrichment of genes belonging to each cluster with respect to hESCs in pluripotent conditions or differentiated in the early embryonic germ layers using previous RNA-Seq data was determined (C. A. Gifford et al. (supra)). The normalized FPKM counts were log 2 transformed after adding a pseudo-count of 1 to all measurements. To obtain the hESC and germ layer specific gene sets, we computed the ratio of each gene's expression level in each sample to the maximum expression level across the remaining three samples, requiring a minimum expression of 1. If the expression was below one in the remaining samples, we set the maximum to 1 directly. The resulting scores were then used to rank order the genes in each sample separately and the top 1000 genes were chosen as sample specific gene set. To compute the gene set activity for each expression cluster at each time point, we simply computed the sum of log 2 and pseudo-count transformed FPKM values across all cluster member genes overlapping with a particular gene set and plotted the resulting cumulative gene set activity at each stage for distinct cluster groups.

Gene ontology analysis from resulting clusters was performed using DAVID pipeline (D. W. Huang et al (2009) Nature Protocols, 4: 44-57) restricted to GO:Biological Processes displaying the top GO terms with FDR <5%, or using LifeMap discovery (R. Edgar et al. (2013) PLoS ONE, 8: e66629) road map of cellular embryonic development displaying for each cluster the cells/compartments showing the highest identity score normalized for the size of each cluster.

Estimation of transcripts expression from human pre- and post-implantation phases was performed using existing RNA-seq Datasets (L. Yan et al. (2013) Nat. Struct. Mol. Biol., 20: 1131-1139). Validation of RNAseq data was performed by taqman-based qPCR (Life Technologies).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A cell that i) contains an inducible first polycistronic exogenous nucleic acid encoding one or more reprogramming factors operably linked to a first regulatory sequence; and ii) expresses a second exogenous nucleic acid encoding an immortalizing factor operably linked to a second regulatory sequence; wherein the cell is capable of proliferating through at least 10, 20, 25, 30, 35, or even 40 or more population doublings.

2. The cell of claim 1, wherein the cell is a human cell or a mouse cell.

3. The cell of claim 1, wherein the cell is a somatic cell, a fibroblast, a keratinocyte or an adult stem cell.

4-6. (canceled)

7. The cell of claim 1, wherein the one or more reprogramming factors are pluripotency factors selected from Oct4, KLF4, Myc, and Sox2, and combinations thereof.

8. The cell of claim 1, wherein the immortalizing factor is hTERT.

9. The cell of claim 1, wherein the cell does not express the reprogramming factors encoded in the inducible first polycistronic exogenous nucleic acid.

10. The cell of claim 1, wherein the second exogenous nucleic acid is capable of being deactivated when the first polycistronic exogenous nucleic acid is expressed, or wherein the second exogenous nucleic acid is inducible.

11. (canceled)

12. A cell produced by inducing expression of the reprogramming factors encoded in the inducible first polycistronic exogenous nucleic acid in a cell of claim 1.

13. A population of substantially uniform cells that i) express an inducible first polycistronic exogenous nucleic acid encoding one or more reprogramming factors operably linked to a first regulatory sequence; and ii) express a second exogenous nucleic acid encoding an immortalizing factor operably linked to a second regulatory sequence; wherein the population optionally comprises at least 4 million, 8 million, 16 million, 32 million, 64 million, or even 128 million or more substantially uniform cells.

14. (canceled)

15. A method of reprogramming a cell, comprising inducing expression of the reprogramming factors encoded in the inducible first polycistronic exogenous nucleic acid in a cell of claim 1; wherein the method is optionally carried out on a population of at least 4 million, 8 million, 16 million, 32 million, 64 million, or even 128 million or more substantially uniform cells.

16. (canceled)

17. A method of producing an immortalized secondary cell, comprising:
i) introducing into an initial cell an inducible first polycistronic exogenous nucleic acid encoding pluripotency factors operably linked to a first regulatory sequence;
ii) inducing expression of the pluripotency factors encoded in the inducible first polycistronic exogenous nucleic acid to produce a de-differentiated cell;
iii) exposing the de-differentiated cell to differentiation agents to produce a secondary cell; and
iv) introducing into the secondary cell a second exogenous nucleic acid encoding an immortalizing factor operably linked to a second regulatory sequence; and
v) causing the secondary cell to express the immortalizing factor;
wherein the method may optionally further comprise proliferating the immortalized secondary cells for at least 10, 15, 20, 25, 30, 35, or even 40 or more population doublings.

18. (canceled)

19. A method of claim 17, further comprising inducing expression of the pluripotency factors encoded in the inducible first polycistronic exogenous nucleic acid in all or a portion of the proliferated secondary cells.

20. (canceled)

21. The method of claim 19, further comprising inducing expression of the pluripotency factors encoded in the inducible first polycistronic exogenous nucleic acid in the secondary cell that expresses or expressed the immortalizing factor, wherein inducing the expression of the pluripotency factors causes the immortalizing factor to stop being expressed.

22-25. (canceled)

26. The method of claim 17, wherein the initial cell is a somatic cell, a fibroblast, or a keratinocyte.

27. (canceled)

28. The method of claim 26, wherein the somatic cell is an adult stem cell, a hematopoietic stem cell, neural stem cell, or mesenchymal stem cell.

29-32. (canceled)

33. A cell produced by the method of claim 15.

34. A method of producing an engineered cell, comprising:
i) introducing into an initial cell an inducible exogenous nucleic acid encoding one or more reprogramming factors operably linked to a first regulatory sequence and an exogenous nucleic acid encoding an immortalizing factor operably linked to a second regulatory sequence;
ii) inducing expression of the one or more reprogramming factors encoded in the inducible exogenous nucleic acid; and
iii) causing the cell to express the immortalizing factor; and
wherein the method optionally further comprises proliferating the cell expressing the immortalizing factor for at least 10, 15, 20, 25, 30, 35, or even 40 or more population doublings.

35-38. (canceled)

39. A method of producing an immortalized secondary cell, comprising:
i) introducing into an initial cell an inducible exogenous nucleic acid encoding one or more reprogramming factors operably linked to a first regulatory sequence;
ii) inducing expression of the reprogramming factors encoded in the inducible exogenous nucleic acid to produce a reprogrammed cell;
iv) introducing into the reprogrammed cell an exogenous nucleic acid encoding an immortalizing factor operably linked to a second regulatory sequence, and
v) causing the reprogrammed cell to express the immortalizing factor; and
wherein the method optionally further comprises proliferating the cell expressing the immortalizing factor for at least 10, 15, 20, 25, 30, 35, or even 40 or more population doublings.

40. A method of producing an immortalized secondary cell, comprising:
i) introducing into an initial cell an exogenous nucleic acid encoding an immortalizing factor operably linked to a first regulatory sequence;
ii) causing the cell to express the immortalizing factor, thereby producing an immortalized cell;
iii) introducing into the immortalized cell an inducible exogenous nucleic acid encoding one or more reprogramming factors operably linked to a second regulatory sequence, and
iv) inducing expression of the one or more reprogramming factors encoded in the inducible exogenous nucleic acid to produce a reprogrammed cell; and
wherein the method optionally further comprises proliferating the cell expressing the immortalizing factor for at least 10, 15, 20, 25, 30, 35, or even 40 or more population doublings.

41. (canceled)

42. A method of claim 34, further comprising inducing expression of the one or more reprogramming factors in the secondary cell that expresses the immortalizing factor or has been induced to express the immortalizing factor, wherein inducing the expression of the one or more reprogramming factors causes the immortalizing factor to stop being expressed.

43-46. (canceled)

47. The method of claim 34, wherein the immortalizing factor is constitutively expressed after being introduced into the cell.

48-55. (canceled)

56. A cell produced by the method of claim 34.

57. A method of identifying an agent that affects nuclear reprogramming, cellular differentiation, cellular proliferation, cellular viability or cellular metabolism comprising exposing a cell of claim 1, or a cell derived therefrom, to the test agent, and detecting, identifying, and/or quantifying a change in nuclear reprogramming, cellular differentiation, cellular proliferation, cellular viability or cellular metabolism, respectively, wherein a change in nuclear reprogramming, cellular differentiation, cellular proliferation, cellular viability or cellular metabolism, respectively relative to an untreated control cell indicates that the test agent affects nuclear reprogramming, cellular differentiation, cellular proliferation, cellular viability or cellular metabolism, respectively; wherein the method is optionally performed as a high-throughput assay in which a plurality of test agents are each tested, individually and/or in various combinations, on a plurality of substantially uniform cells.

58-63. (canceled)

64. A method of identifying an agent that affects a cellular characteristic comprising exposing a cell of claim 1, or a cell derived therefrom, to the test agent, and detecting, identifying, and/or quantifying a change in the cellular characteristic, wherein a change in the cellular characteristic relative to an untreated control cell indicates that the test agent affects the cellular characteristic;
wherein the method is optionally performed as a high-throughput assay in which a plurality of test agents are each tested, individually and/or in various combinations, on a plurality of substantially uniform cells.
